(19) United States
(12) Patent Application Publication
(10) Pub. No.: US 2016/0324447 A1
(43) Pub. Date: Nov. 10, 2016

(54) System and Method for Determining Orientation of Body Segments Using Inertial Measurement Units

(71) Applicant: Sharp Laboratories of America (SLA), Inc., Camas, WA (US)

(72) Inventor: Bryan Hallberg, Vancouver, WA (US)

(21) Appl. No.: 15/091,869

(22) Filed: Apr. 6, 2016

Related U.S. Application Data
(63) Continuation-in-part of application No. 14/873,946, filed on Oct. 2, 2015.
Application No. 14/873,946 is a continuation-in-part of application No. 14/742,852, filed on Jun. 18, 2015.
Application No. 14/742,852 is a continuation-in-part of application No. 14/707,194, filed on May 8, 2015, now Pat. No. 9,450,681.

(51) Int. Cl. A61B 5/11; G01B 5/24; G01P 15/02; G01B 7/30

(52) U.S. Cl. CPC A61B 5/1122; G01B 7/30; G01B 5/24; G01P 15/02

(57) ABSTRACT

A system and method is provided for determining the orientation of a body segment using an inertial measurement unit (IMU) sensor capable of measuring its orientation relative to Earth. In general, the method mounts a primary IMU sensor on a first body segment, with an unknown first alignment orientation relationship between the primary IMU sensor and the first body segment. A primary IMU sensor orientation is measured, and an alignment orientation relationship is calculated between the primary IMU sensor orientation and a first body segment orientation. The method may also measure a primary IMU sensor initial orientation and a subsequent orientation. As a result, a subsequent orientation of the first body segment is determined based upon the primary IMU sensor initial and subsequent orientations, as well as the calculation of the alignment orientation relationship between the primary IMU sensor initial orientation and the first body segment orientation.

### BACKGROUND OF THE INVENTION

1. Field of the Invention

This invention generally relates to position location and, more particularly, to a system and method for determining the orientation of body segments using an inertial measurement unit (IMU).

2. Description of the Related Art

Measuring the orientation of human body segments is critical for applications such as joint surgery recovery, sports technique coaching, or motion monitoring. IMUs can be placed on body segments to measure their orientation. However, the IMUs actually report the orientation of themselves and the local epidermis surface orientation, which generally is not the same as the orientation of the body segment's major axis. For example, if the IMU is attached to the foot, the angle of the foot's top affects the IMUs orientation measurement. Similarly, if the IMU resides on muscle or body fat, the muscle's or fat's curvature affects the IMU's reading. In general, sensors can also be randomly oriented on body segments, producing a random and uncorrelated offset to their readings.

Currently existing systems either assume that the sensor's orientation is identical to the body segment's orientation, or use multiple sensors on a single segment to estimate the segment's orientation by averaging the readings produced by each sensor. These systems also assume a known location for the sensor on a body segment.

FIG. 1 is a diagram depicting an exemplary difference between radial epidermis angle and body segment major axis. Estimating the orientation of a body segment and the relative direction the segment is facing based on the orientation of an attached IMU can be error prone due to the randomness of where a sensor is placed on a body segment and the angle of the segment's epidermis relative to the segment's actual major axis. For example, sensors placed on a leg can easily report an offset from each leg segment's radial orientation. Ideally, a user would place a sensor on a relatively flat location of the body segment, but this cannot be guaranteed, and may not even be possible. As shown in the example, the thigh sensor reports an offset of 23 degrees from the thigh's major axial axis. The shank also reports a non-zero offset. When combined, the two sensors would report that the knee is bent at 57 degrees (90−23−10), rather than the actual 90 degrees at which it is bent. Further, these measurements also assume that the sensors are all placed on the front or back of the limb, while the user often places them randomly, based on ease of attachment or comfort of wearing.

FIG. 2 is a diagram depicting an exemplary difference between actual and measured axial orientation. Relative axial orientation can also suffer from offset measurement. For example, the rotation of the hand relative to the forearm shows an offset of 73 degrees due to the actual location of the sensor relative to the expected location of the sensor. Requesting a user to accurately place sensors relative to each other is not practical, so methods to compensate for this random sensor placement case must be developed.

FIG. 3 is a diagram depicting a global motion error based upon sensor orientation. Relative sensor orientation also affects global motion estimation. For example, the figure of the left shows the actual orientation of a sensor on a user's foot from the perspective of looking at the user from overhead. As the user moves their foot forward, the sensor measures motion in the sensor's X-axis direction. However, if the system expects the sensor to have been placed with the orientation shown on the right figure, then the system would interpret the motion to have been caused by the foot moving to the right.

To compensate for the above issues, the actual orientation of the sensor relative to its associated body segment must be determined. That determination must be able to be performed quickly and easily when using both few and multiple sensors.

It would be advantageous if the orientation of body segments could be accurately determined using only a single sensor per body segment, and still account for body segment epidermal curvature relative to the segment's major axis orientation. It would also be advantageous if accurate orientation could be maintained by accounting for the random placement of the sensor on the body segment.

### SUMMARY OF THE INVENTION

Disclosed herein are a system and method that provides for the estimation of body segment orientation relative to each other and to a reference object. Each of the segments have an inertial measurement unit (IMU) attached to them reporting the orientation of the IMU relative to a reference object that emits gravitational and magnetic fields, such as Earth. Sensor orientation updates may be received from any number of sensors. Multiple methods are presented for determining the orientation of the associated body segments.

For example, one method is to have the user pose in a predetermined position, such as standing, then have the user point in the direction they are facing. This method works well for healthy people with multiple sensors. However, for users assessing range of motion for a single joint with minimal mobility, the initial body segment alignment needs to be determined when the user potentially cannot assume a predetermined pose and has little mobility to indicate the direction a body segment is facing. Different alignment methodologies also present different user interface fields to enter the necessary data to map sensor alignment to body segment alignment.

In general, many alignment estimation techniques start by determining an initial user pose, then track alignment changes from that pose. Methods of measuring initial pose include having the user assume a predetermined pose, using a three-dimensional (3D) body segment orientation measurement device, and using a goniometer. Body segment orientation can also be estimated without knowing the initial pose by tracking the user's movements and correlating those movements to a musculoskeletal model with range of motion metrics.

One method for determining the alignment of IMU sensors on a user has the user assume a predetermined pose in a predetermined direction and press a button on a user interface. Detecting when the user is still, the method captures initial sensor orientations, and computes new body segment orientations based upon the initial pose, initial direction, initial sensor readings, and future sensor readings.

One method for determining alignment of IMU sensors on a user has the user assume a predetermined pose in an arbitrary direction, align an Earth relative orientation measurement device (EROMD) with a reference body segment, and press a button on a user interface. The method captures the orientation of the EROMD and reference body segment, detects when the user is still, captures initial sensor orientations, calculates the user's initial orientation from the EROMD and initial sensor values, and computes new body segment orientations based upon the initial pose, captured EROMD orientation, reference body segment orientation, initial sensor readings, and future sensor readings.

Another method for determining alignment of IMU sensors on a user has the user assume a predetermined pose in an arbitrary direction and press a button on a user interface. The method detects when the user is still, captures initial sensor orientations, prompts the user to perform a predetermined move, detects when the user has completed the move, calculates the user's initial orientation from the predetermined move, and computes new body segment orientations based upon the initial pose, predetermined move, initial sensor readings, and future sensor readings.

One method for determining alignment of IMU sensors on a user who is in an arbitrary pose prompts the user to align an EROMD with each body segment of interest and capture the orientation of the body segment and the EROMD. The method computes new body segment orientations based upon the EROMD measurements, initial sensor readings, and future sensor readings.

Another method for determining alignment of IMU sensors on a user who is an arbitrary pose prompts the user to align a goniometer with a body segment of interest and an auxiliary body segment of known orientation, and enter the goniometer reading into an application. The method captures the sensor reading when the goniometer is aligned with the body segments and computes new body segment orientations based upon the goniometer measurement, auxiliary segment orientation, initial sensor readings, and future sensor readings.

One method for determining alignment of IMU sensors on a user who is an arbitrary pose estimates the sensor offset from the body segment based upon a physiological model of the body and adjusts calculated sensor offsets based upon the measured relative orientation of sensors on adjoining limbs in comparison to the physiological model. One method for decomposing the relative orientation of adjoining body segments into an axial and a radial rotation is based upon a physiological model that favors physiologically possible joint rotations. Further, comparing relative adjoining estimated body segment orientations to a physiological model permits the user to be alerted when the joint rotations exceed the joint rotation limits of the model as this may be an indication that the sensors have moved from their initial positions on the user.

Accordingly, a method is provided for determining the orientation of a body segment using an IMU sensor capable of measuring its orientation relative to Earth. In general, the method mounts a primary IMU sensor on a first body segment, with an unknown first alignment orientation relationship between the primary IMU sensor and the first body segment. A primary IMU sensor orientation is measured, and an alignment orientation relationship is calculated between the primary IMU sensor orientation and a first body segment orientation. The method may also measure a primary IMU sensor initial orientation and a subsequent orientation. As a result, a subsequent orientation of the first body segment is determined based upon the primary IMU sensor initial and subsequent orientations, as well as the calculation of the alignment orientation relationship between the primary IMU sensor orientation and the first body segment orientation.

In one aspect, determining the subsequent orientation of the first body segment includes using a body segment musculoskeletal model describing potential movement relationships between adjacent body segments to find deterministic, axial rotation, and radial rotation limits associated with the first body segment, where deterministic limits describe the likely accuracy of a first alignment orientation relationship estimate. The method alerts a user when the estimated relationship between adjacent body segments exceeds the limits of the musculoskeletal model.

In another aspect, the primary IMU sensor measures its orientation with the first body segment in a predetermined pose, aligned in a predetermined direction relative to Earth. In another aspect, an EROMD is aligned with a predetermined second body segment. Then, simultaneous with measuring the primary IMU sensor orientation, the method measures an EROMD orientation with the second body segment in a predetermined pose, in an arbitrary direction relative to Earth. In a variation, an EROMD is aligned with the first body segment. Simultaneous with measuring the primary IMU sensor orientation, the EROMD orientation is measured with the first body segment in an arbitrary pose, in an arbitrary direction relative to Earth.

In another variation, the primary IMU sensor measures the first body segment orientation with respect to a second body segment using a goniometer. Then, simultaneous with measuring the primary IMU sensor orientation, the method measures the orientation of an auxiliary IMU sensor mounted on the second body segment, where an alignment orientation relationship between the auxiliary IMU sensor and second body segment is known.

In one aspect, the primary IMU sensor measures a primary IMU sensor first orientation with the first body segment in a predetermined pose, aligned in an arbitrary direction relative to Earth, and then measures a primary IMU sensor second orientation with the first body segment moving in a predetermined manner.

In another aspect, the alignment orientation relationship between the primary IMU sensor orientation and the first body segment orientation is estimated. Then, the body segment musculoskeletal model is used. In response to comparing the deterministic, axial rotation, and radial rotation limits with subsequent calculated movement relationships between the first body segment and an adjacent body segment, the estimated alignment orientation relationship is updated.

Additional details of the above-described method and a system for determining the orientation of a body segment using an IMU sensor are provided below.

### BRIEF DESCRIPTION OF THE DRAWINGS

FIG. 1 is a diagram depicting an exemplary difference between radial epidermis angle and body segment major axis.

FIG. 2 is a diagram depicting an exemplary difference between actual and measured axial orientation.

FIG. 3 is a diagram depicting a global motion error based upon sensor orientation.

FIG. 4 is a schematic block diagram depicting a system for determining the orientation of a body segment using an inertial measurement unit (IMU) sensor capable of measuring its orientation relative to Earth.

FIG. 5 is a diagram of a variation of body segment orientation determination using an auxiliary IMU sensor.

FIGS. 6A and 6B are, respectively, coordinate systems for an IMU sensor, and an IMU sensor as mounted on a body segment.

FIG. 7 shows sensor orientations for each body segment when sensors are placed on a user that is standing facing Earth south with their arms at their sides and thumbs facing forward.

FIG. 8 is a drawing supporting an overview and explanation of quaternions.

FIG. 9 depicts a relative body segment orientation change.

FIG. 10 is a flowchart illustrating a body segment orientation determination method based upon using a predetermined pose and a predetermined direction.

FIG. 11 is a flowchart illustrating a body segment orientation determination method based upon using a predetermined pose and an unknown direction with the aid of an EROMD.

FIG. 12 is a flowchart illustrating a body segment orientation determination method based upon using a predetermined pose and an unknown direction, making a predetermined move.

FIG. 13 is a flowchart illustrating an arbitrary pose method for the determination of body segment orientation using an EROMD.

FIG. 14 is a flowchart illustrating an arbitrary pose method of determining body segment orientation using a goniometer.

FIG. 15 is a table listing some exemplary parameters used in the musculoskeletal model, and exemplary values for body segments.

FIGS. 16A and 16B are a flowchart illustrating an arbitrary pose, musculoskeletal model for determining body segment orientation.

FIG. 17 is a continuous elliptical model of joint rotation.

FIGS. 18A and 18B are a flowchart summarizing the above-described method for determining the orientation of a body segment using an IMU sensor capable of measuring its orientation relative to Earth.

FIG. 19 is a flowchart illustrating a method for determining separate constituent axial and radial rotations of a connected joint.

### DETAILED DESCRIPTION

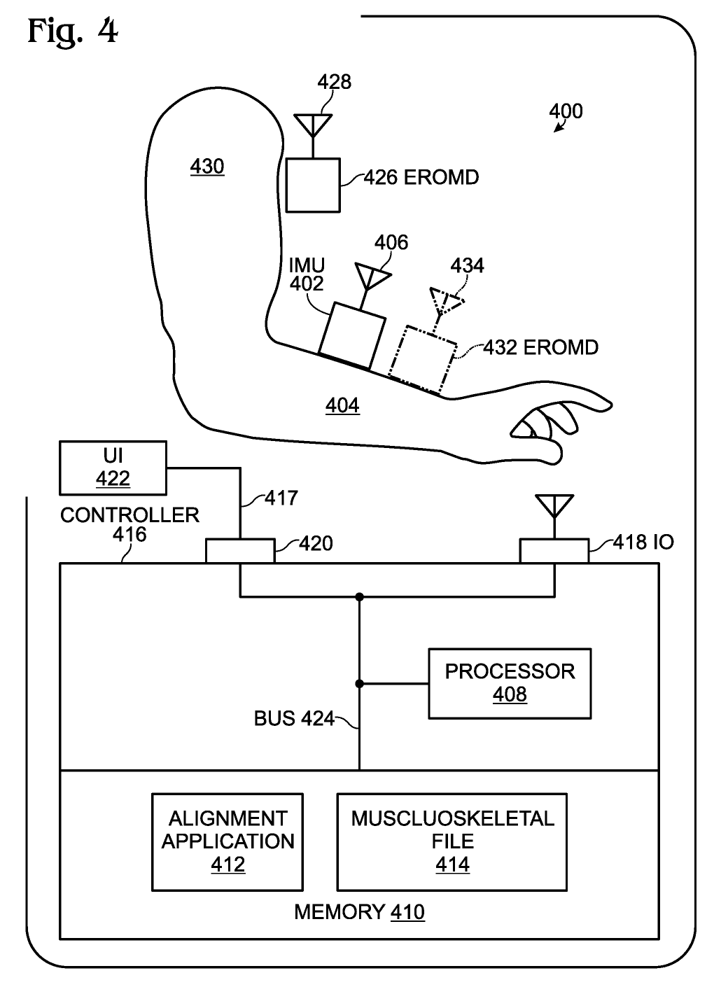

FIG. 4 is a schematic block diagram depicting a system for determining the orientation of a body segment using an inertial measurement unit (IMU) sensor capable of measuring its orientation relative to Earth. The system 400 comprises a primary IMU sensor 402 mounted on a first body segment 404 and having an output 406 to supply signals associated with an unknown first alignment orientation relationship between the primary IMU sensor and the first body segment. The system 400 further comprises a processor 408, a non-transitory memory 410, and an alignment application 412 embedded in the non-transitory memory including a sequence of processor executable instructions. The alignment application 412 accepts the primary IMU sensor signals, measures a primary IMU sensor orientation, and calculates an alignment orientation relationship between the primary IMU sensor orientation and a first body segment orientation.

As shown, the IMU output 406 is enabled as a wireless device, however, in some circumstances the output may be a hardwired or be an optical interface. The figure also implies that the processor 408, memory 410, and alignment application 412 reside in an external device, which for convenience may be termed a controller or central collection hub 416. In this case, wireless communications may be received via input/output (IO) port 418. For example, the controller 416 may be a smartphone, personal computer, or stand-alone device. However, in some aspects the processor 408, memory 410, and alignment application 412 reside in the IMU 402, in which case the IMU output would be internal.

In one aspect, the alignment application 412 measures a primary IMU sensor initial orientation and a subsequent orientation. The alignment application 412 determines a subsequent orientation of the first body segment in response to the primary IMU sensor initial and subsequent orientations, as well as in response to the calculation of the alignment orientation relationship between the primary IMU sensor orientation and the first body segment orientation.

In one aspect, a body segment musculoskeletal model (file) 414 is stored in the non-transitory memory 410, which describes potential movement relationships between adjacent body segments. In this case the alignment application 412 determines the subsequent orientation of the first body segment using the musculoskeletal model to find deterministic, axial rotation, and radial rotation limits associated with the first body segment. As used herein, deterministic limits describe the likely accuracy of a first alignment orientation relationship estimate. In another aspect, the body segment musculoskeletal model 414 describes physiologically possible constituent rotations for a first joint connecting two adjoining body segments, and the alignment application 412 determines separate constituent axial and radial rotations for the first joint by applying the musculoskeletal model.

The alignment application 412 has an interface on line 417 for alerting a user when the estimated relationship between adjacent body segments exceeds the limits of the musculoskeletal model. Line 417 may connect IO port 420 to a user interface 422, such as a monitor or display device, keyboard, keypad or a cursor control device such as a mouse, touchpad, touchscreen, trackball, stylus, cursor direction keys, or other means for a user to enter commands and receive information.

In one variation, the alignment application 412 estimates the alignment orientation relationship between the primary IMU sensor 402 orientation and the first body segment orientation 404. The alignment application 412 calculates the alignment orientation relationship between the primary IMU sensor orientation and the first body segment orientation by using the body segment musculoskeletal model to find deterministic, axial rotation, and radial rotation limits associated with the first body segment. As above, deterministic limits describe the likely accuracy of the estimated alignment orientation relationship. The alignment application 412 compares the deterministic, axial rotation, and radial rotation limits with subsequent calculated movement relationships between the first body segment 404 and an adjacent body segment 430, and updates the estimated alignment orientation relationship.

The controller 416 typically uses a communications bus 424. The communication bus 424 may, for example, be a Serial Peripheral Interface (SPI), an Inter-Integrated Circuit (I²C), a Universal Asynchronous Receiver/Transmitter (UART), and/or any other suitable bus or network. Although the drawing implies that the components of the controller 416 are collocated in the same device, in some aspects various components may be located outside the device, communicating with other components via a hardwire or wireless connection.

The memory 410 may include a main memory, a random access memory (RAM), or other dynamic storage devices. These memories may also be referred to as a computer-readable medium. Such a medium may take many forms, including but not limited to, non-volatile media, volatile media, and transmission media. Non-volatile media includes, for example, optical or magnetic disks. Volatile media includes dynamic memory. Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, or any other medium from which a computer can read. The execution of the sequences of instructions contained in a computer-readable medium (i.e. alignment application 412) may cause the processor 408 to perform some of the steps of determining IMU sensor and body segment alignment. Alternately, some of these functions may be performed in hardware (not shown). The practical implementation of such a computer system would be well known to one with skill in the art. In one aspect, the processor 408 is a 16-bit microcontroller or an ARM processor using a reduced instruction set computing (RISC) architecture.

The IO ports 418 and 420 may incorporate a modem, an Ethernet card, or any other appropriate data communications device such as USB. The physical communication links may be optical, wired, or wireless. The controller 416 may be considered a type of special purpose computing system, and as such, can be programmed, configured, and/or otherwise designed to comply with one or more networking protocols. According to certain embodiments, the controller 416 may be designed to work with protocols of one or more layers of the Open Systems Interconnection (OSI) reference model, such as a physical layer protocol, a link layer protocol, a network layer protocol, a transport layer protocol, a session layer protocol, a presentation layer protocol, and/or an application layer protocol. For example, IOs 418 and 420 may include a network device configured according to a Universal Serial Bus (USB) protocol, an Institute of Electrical and Electronics Engineers (IEEE) 1394 protocol, an Ethernet protocol, a T1 protocol, a Synchronous Optical Networking (SONET) protocol, a Synchronous Digital Hierarchy (SDH) protocol, an Integrated Services Digital Network (ISDN) protocol, an Asynchronous Transfer Mode (ATM) protocol, a Point-to-Point Protocol (PPP), a Point-to-Point Protocol over Ethernet (PPPoE), a Point-to-Point Protocol over ATM (PPPoA), a Bluetooth protocol, an IEEE 802.XX protocol, a frame relay protocol, a token ring protocol, a spanning tree protocol, and/or any other suitable protocol.

The controller 416 may provide a direct connection to a remote server via a direct link to a network, such as the Internet. Connection may be provided through, for example, a local area network (such as an Ethernet network), a personal area network, a wide area network, a private network (e.g., a virtual private network), a telephone or cable network, a cellular telephone connection, a satellite data connection, or any other suitable connection.

In certain embodiments, a host adapter is configured to facilitate communication between controller 416 and one or more network or storage devices via an external bus or communications channel. Examples of host adapters include, without limitation, Small Computer System Interface (SCSI) host adapters, Universal Serial Bus (USB) host adapters, IEEE 1394 host adapters, Advanced Technology Attachment (ATA), Parallel ATA (PATA), Serial ATA (SATA), and External SATA (eSATA) host adapters, Fibre Channel interface adapters, Ethernet adapters, or the like.

In one aspect, the alignment application 412 measures the primary IMU sensor orientation with the first body segment in a predetermined pose, aligned in a predetermined direction relative to Earth. For example, the first body segment may be a right forearm vertically extended down from the user, with the forearm's under side facing the user and its top side facing the direction West.

In another aspect, an Earth relative orientation measurement device (EROMD) 426 has an output 428 to supply signals associated with its current orientation relative to Earth. The EROMD is aligned with a predetermined second body segment 430 in a predetermined pose, in an arbitrary direction relative to Earth. For example, the second body segment 430 may be an upper arm extending vertically in front of the user, but without the user knowing the direction in which they are standing. In this case the alignment application 412, simultaneous with measuring the primary IMU sensor orientation, measures the EROMD orientation. In one variation, the EROMD and controller may be the same device.

An EROMD is a device with visible alignment markings and containing an IMU and a communication mechanism. The alignment markings enable a user to align the EROMD with a body segment. Alignment markings can come in many forms, for example, lines or grids drawn on the surface of the device in 3D orthogonal orientations, or thin light beams emitted from the device as lines or grids in 2D or 3D orthogonal orientations that can be imaged onto the surface of the body segment.

In a related aspect, an EROMD 432 (in phantom) has an output 434 to supply signals associated with its current orientation relative to Earth, aligned with the first body segment 404 in an arbitrary pose, in an arbitrary direction relative to Earth. In this case, the alignment application 412 simultaneously measures the primary IMU sensor orientation and the EROMD orientation.

In another aspect, the alignment application 412 measures a primary IMU sensor first orientation with the first body segment in a predetermined pose, aligned in an arbitrary direction relative to Earth, and measures a primary IMU sensor second orientation with the first body segment moving in a predetermined manner. For example, a right forearm vertically extended down from the user, with the forearm's under side facing the user and its top side facing an unknown direction relative to Earth. The predetermined movement may be the user lifting their forearm so that it is “pointing” horizontally forward.

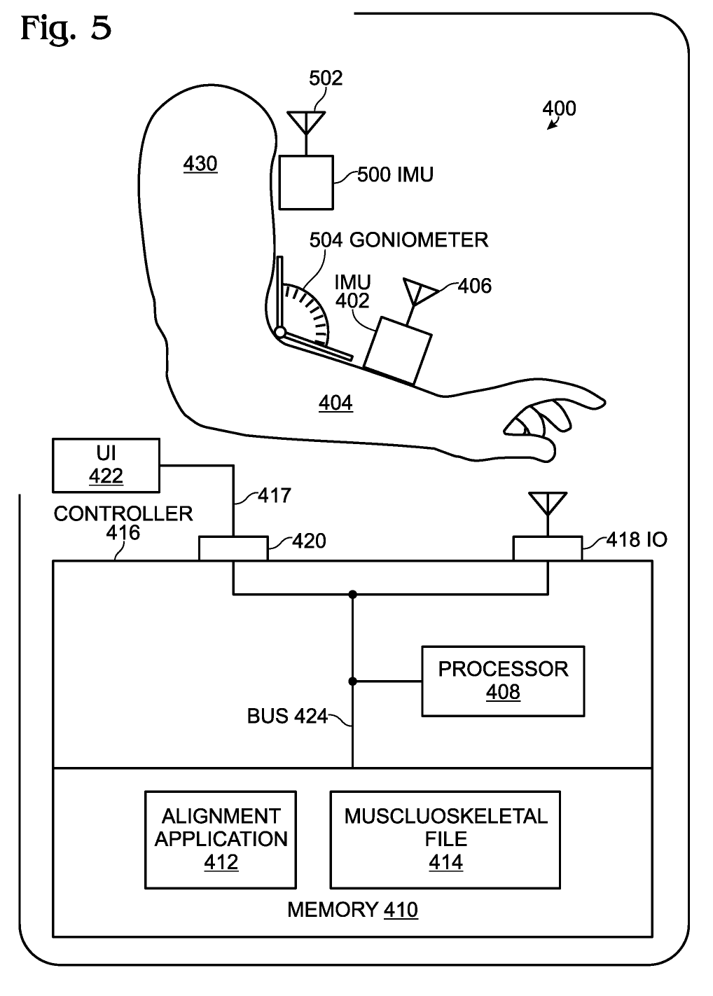

FIG. 5 is a diagram of a variation of body segment orientation determination using an auxiliary IMU sensor. An auxiliary IMU sensor 500 has an output 502 to supply signals associated with being mounted on a second body segment 430, where an alignment orientation relationship between the auxiliary IMU sensor and second body segment is known. The alignment application 412 has an interface on line 417 to accept a measurement of the first body segment orientation with respect to a second body segment found using a goniometer 504. In this case, the alignment application 412 calculates the primary IMU sensor 402 orientation relationship by simultaneously measuring the primary IMU sensor orientation and the auxiliary IMU sensor 500 orientation.

In a related aspect, the orientation of the second body segment 430 can also be determined. The auxiliary IMU sensor 500 is mounted on the second body segment 430 with an unknown second alignment orientation relationship between the auxiliary IMU sensor and second body segment. The second body segment 430 assumes a predetermined pose, aligned in an arbitrary direction relative to Earth. In this case, the alignment application 412, simultaneous with measuring the primary IMU sensor's first orientation, measures the auxiliary IMU sensor orientation, and calculates an auxiliary alignment orientation relationship between the auxiliary IMU sensor orientation and the second body segment orientation.

The goal of all the above-described system is to determine the orientation of each body segment relative to each other and Earth, and to accurately calculate body segment orientation independent of sensor placement location and orientation on body segments. The body segment orientation calculations are based on readings from IMU based sensors placed on the body segments. Each individual sensor independently calculates its own orientation and reports it to a central collection hub (controller) that synthesizes the data into a final body segment orientation output. The orientation can be reported in numerous forms, independent of the usage of that data in this disclosure. For example, it could be reported as a rotational matrix or as a quaternion. Parent application Ser. No. 14/873,946, entitled, SYSTEM AND METHOD FOR DETERMINING THE ORIENTATION OF AN INERTIAL MEASUREMENT UNIT (IMU), filed Oct. 2, 2015, describes a method for calculating an IMU's orientation. Parent application Ser. No. 14/707,194, entitled, METHOD AND SYSTEM FOR WIRELESS TRANSMISSION OF QUATERNIONS, filed May 8, 2015, describes a method for transmitting orientation data in quaternion form.

FIGS. 6A and 6B are, respectively, coordinate systems for an IMU sensor, and an IMU sensor as mounted on a body segment. The orientation of objects is referenced to a coordinate system. The figures describe the coordinate systems of relevance for identifying a body segment's orientation relative to Earth, the sensor, and other body segments. Any linearly independent coordinate alignment is possible for any of the coordinate systems listed above. The alignments described here are simply a convenient alignment for measuring body segment orientation. Each sensor reports its orientation relative to the Earth coordinate system. The Earth coordinate system aligns the X-axis with south, the Y-axis with east, and the Z-axis with up. The sensor coordinate system is aligned with the major (Y-axis), middle (X-axis), and minor (Z-axis) axes of the sensor.

Each body segment has an independent coordinate system aligned with that segment. By convention, this body segment coordinate system has its X-axis aligned parallel to the major radial rotational axis of the body segment relative to its proximal body segment, its Y-axis aligned with the axial body segment axis, and its Z-axis aligned perpendicular to the body segment's major radial rotational axis. For convenience, four often used sensor orientations relative to a body segment are labeled south, east, west, and top.

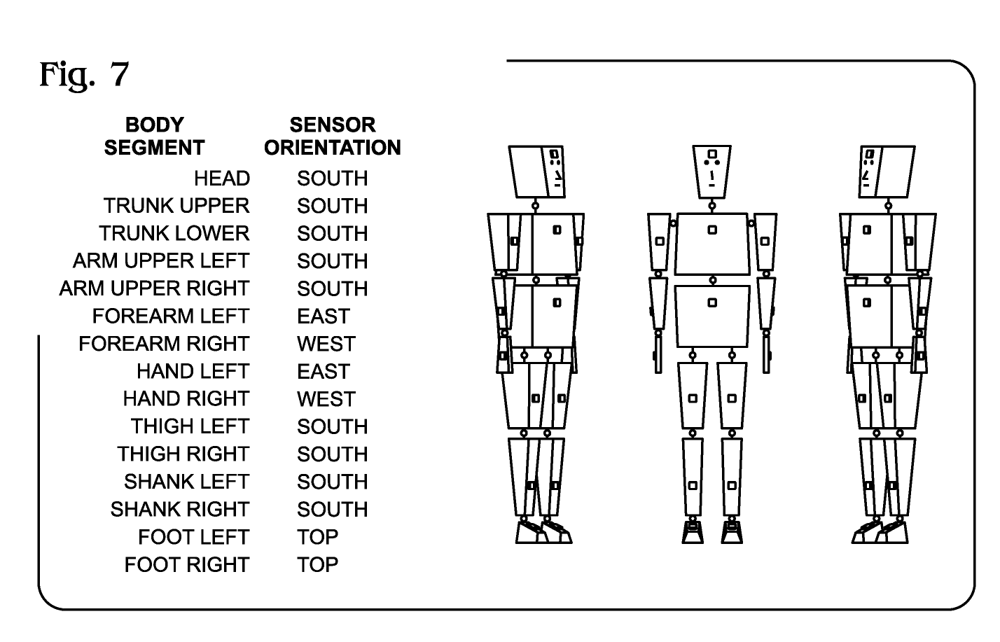

FIG. 7 shows sensor orientations for each body segment when sensors are placed on a user that is standing facing Earth south with their arms at their sides and thumbs facing forward. The notation _{E}^{P}B means the orientation of the body segment (B) for a pose (P) relative to Earth (E).

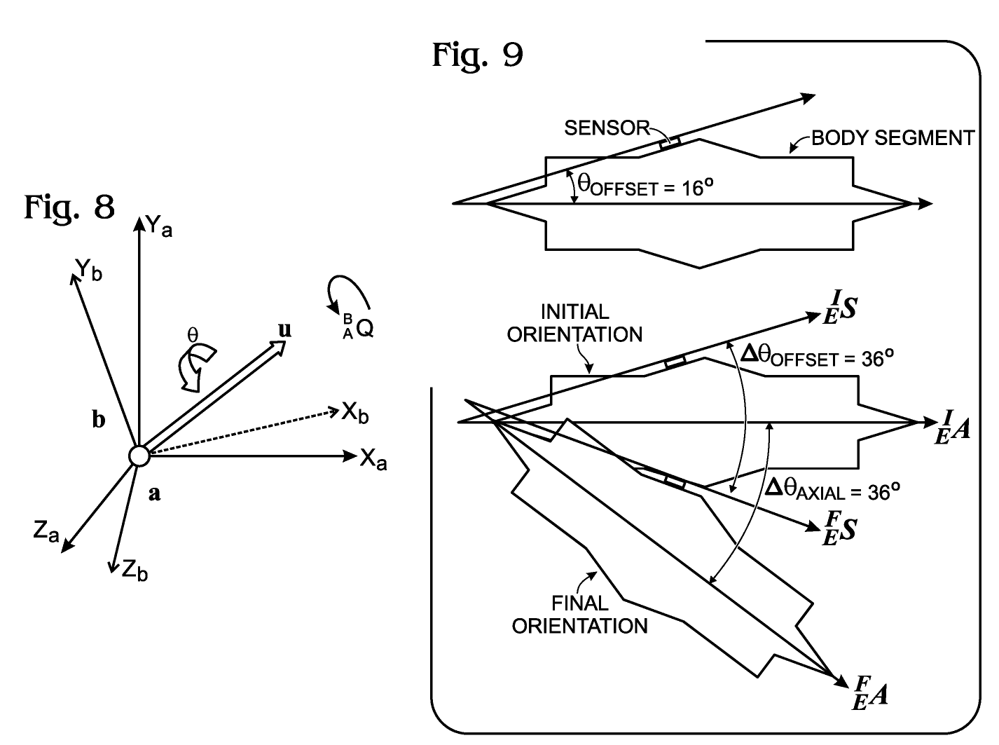

FIG. 8 is a drawing supporting an overview and explanation of quaternions. A quaternion is a 4-element vector (r, x, y, z) based on rotation angle θ and unit length rotation axis [symbol] . A quaternion that rotates an object from spatial coordinate system A to spatial coordinate system B is defined as the normalized vector _{A}^{B}Q.

$${}_{A}^{B} Q = ( r , x , y , z ) = r + \hat{i} x + \hat{j} y + \hat{k} z$$

$${\hat{i}}^{2} = {\hat{j}}^{2} = {\hat{k}}^{2} = \hat{i} \hat{j} \hat{k} = - 1$$

$$r = cos ( \frac{θ}{2} ) ; x = {u}_{x} sin ( \frac{θ}{2} ) ; y = {u}_{y} sin ( \frac{θ}{2} ) ; z = {u}_{z} sin ( \frac{θ}{2} )$$

$${r}^{2} + {x}^{2} + {y}^{2} + {z}^{2} = 1$$

### Null Rotation

θ=0

N=(1,0,0,0)

### Inverse of a Given Quaternion

_{A}^{B}Q′=_{B}^{A}Q

θ→−θ;(r,x,y,z)→(r,−x,−y,−z)

### Multiplication

Q₁Q₂=(r₁+îx₁+ĵy₁+\hat{k}z₁)(r₂+îx₂+ĵy₂+\hat{k}z₂)

Q₁Q₂=(r₁r₂−x₁x₂−y₁y₂−z₁z₂)+\hat{i}(r₁x₂+x₁r₂+y₁z₂−z₁y₂)+\hat{j}(r₁y₂−x₁z₂+y₁r₂+z₁x₂)+\hat{k}(r₁z₂+x₁y₂−y₁x₂+z₁r₂)

### Concatenated Rotations

Concatenated rotations are calculated by multiplying quaternions in order of rotations

_{A}^{C}Q=_{B}^{C}Q_{A}^{B}Q

Rotating a Vector from Frame A to Frame B

V_{B}=_{A}^{B}QV_{AA}^{B}Q′=_{A}^{B}QV_{AB}^{A}Q

Additionally, it is often useful to ensure that a specific quaternion element is non-negative, while not altering the actual rotation performed by the quaternion. For example, ensuring that the y element is non-negative in a quaternion that has its x and z elements set to zero causes the r element to definitely represent a rotation about the positive Y-axis of the coordinate system. This operation of ensuring a particular element is non-negative is performed by the positive definite function, PosDef(element, Q).

Qₚ=PosDef(element, Q₀)

Q₀=original quaternion

Qₚ=positive definite quaternion

element=r, x, y, or z

If selected element of Q₀≧0, then {rₚ=r₀; xₚ=x₀; yₚ=y₀; zₚ=z₀}

else {rₚ=−r₀; xₚ=−x₀; yₚ=−y₀; zₚ=−z₀}

Similarly, it can also be useful to have a negative definite value. This is calculated with the NegDef(element, Q) function which inverts the sign of each element when the selected element's value is greater than zero.

FIG. 9 depicts a relative body segment orientation change. As described above, a body segment's orientation is generally not the same as its associated sensor's orientation. However, the change in orientation for the body segment and the sensor are the same. The example shows that when the orientation of a segment changes by a given amount, the orientation of the segment's sensor changes by the same amount. The change in orientation of an object is calculated by multiplying its current orientation by the inverse of its initial orientation. For example:

_{E}^{I}B=body segment initial relative to Earth

_{E}^{F}B=body segment final relative to Earth

_{E}^{I}S=sensor initial relative to Earth

_{E}^{F}S=sensor final relative to Earth

_{I}^{F}B=_{E}^{F}B_{E}^{I}B′=body segment relative change

_{I}^{F}S=_{E}^{F}S_{E}^{I}S′=sensor relative change

_{I}^{F}B=_{E}^{F}B_{E}^{I}B′=_{E}^{F}S_{E}^{I}S′=_{I}^{F}S

Since the sensor's orientation relative to a constant reference (such as Earth) is provided by the sensor's reading, and the body segment's orientation change is the same as the sensor's orientation change, the main issue in determining a body segment's orientation relative to the reference is to determine the body segment's initial orientation relative to the reference, _{E}^{I}B. Given these values, the current body segment orientation can be calculated by the equation:

_{E}^{F}B=_{I}^{F}B_{E}^{I}B=_{I}^{F}S_{E}^{I}B=_{E}^{F}S_{E}^{I}S′_{E}^{I}B

The orientation of a body segment is calculated as data arrives from that segment's associated sensor. Neighboring segment orientations may also be used to calculate the current segment's orientation. For calculations, the segments are distinguished by the labels “current” and “neighbor”.

The process of deriving a body segment's orientation relative to sensor mounted on the body segment is called alignment. Numerous alignment methods are possible to use. The method selected depends upon the particular use case. For example, the quickest and most accurate method is for the user to assume a known pose (such as standing) and then measure the orientation of each body segment's sensor relative to the reference coordinate system. However, this method assumes the user can assume a known pose, which might not be possible if the user has limited mobility. For example, if they are recovering from knee surgery.

Alignment estimation methods divide broadly into two categories. The first category is when the user is initially in a known pose. The application then calculates all future body segment orientations from that initial pose. The second category is when the user is initially in an arbitrary pose, and the method estimates each body segment's orientation based upon a musculoskeletal model of the body and the relative orientation of each sensor as time passes.

As the user moves, it is possible that a sensor may slip and move relative to the body segment to which it is attached. This would produce an error in the estimation of future body segment orientations. To compensate for this error, the joint rotation between two neighboring body segments can be calculated and compared to a musculoskeletal model to determine if the body segment orientation estimates are within physiologically possible movements. If they are not, then the user can be prompted to perform the alignment process again. The method for calculating joint rotation and comparing it to a musculoskeletal model is described herein in the section titled “Arbitrary Pose Method 3: Arbitrary pose, musculoskeletal model”.

Many of the Known Pose alignment methods described below prompt the user to remain still during the alignment process. Still in this context is defined as all sensors have an orientation rate of change less than a predetermined amount, for example 1 degree per second, for a predetermined amount of time, for example 1 second.

### Known Pose Method 1: Predetermined Pose, Predetermined Direction

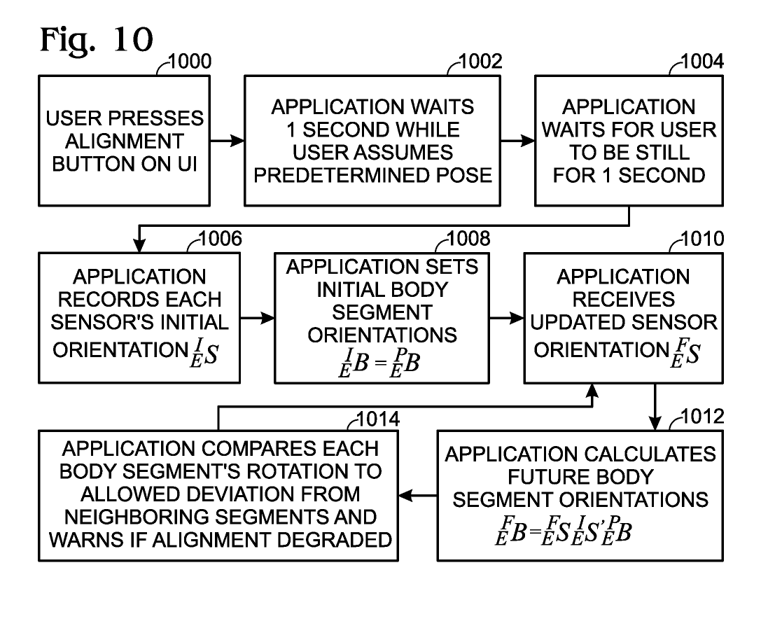

FIG. 10 is a flowchart illustrating a body segment orientation determination method based upon using a predetermined pose and a predetermined direction. In this case, the initial orientation of each individual body segment can be read from a table, such as the one shown in FIG. 7. Other predetermined poses could be used as an initial pose, such as sitting. The user presses a button on the application's user interface (Step 1000) and then assumes a predetermined pose (such as standing) and faces a predetermined direction (such as south). The application waits for the user to move to the predetermined pose (Step 1002) and remain still (Step 1004). Then the application records the initial orientation of each sensor, _{E}^{I}S in Step 1006. In Step 1008 the application sets each body segment's initial orientation equal to its pose orientation:

_{E}^{I}B=_{E}^{P}B

Sensor updates are received in Step 1010, and in Step 1012 the future orientation of each body segment is given by:

_{E}^{F}B=_{I}^{F}S_{E}^{I}B=_{E}^{F}S_{E}^{I}S′_{E}^{P}B

In Step 1014 a comparison is made between body segment movement and allowed deviations with respect to connected body segments. Optionally, this method can compare future estimated body segment joint rotations to a musculoskeletal model and alert the user if the joint rotation is not within physiologically possible movements so that the user may perform the alignment process again.

### Known Pose Method 2: Predetermined Pose, Unknown Direction, Earth Relative Orientation Measurement Device

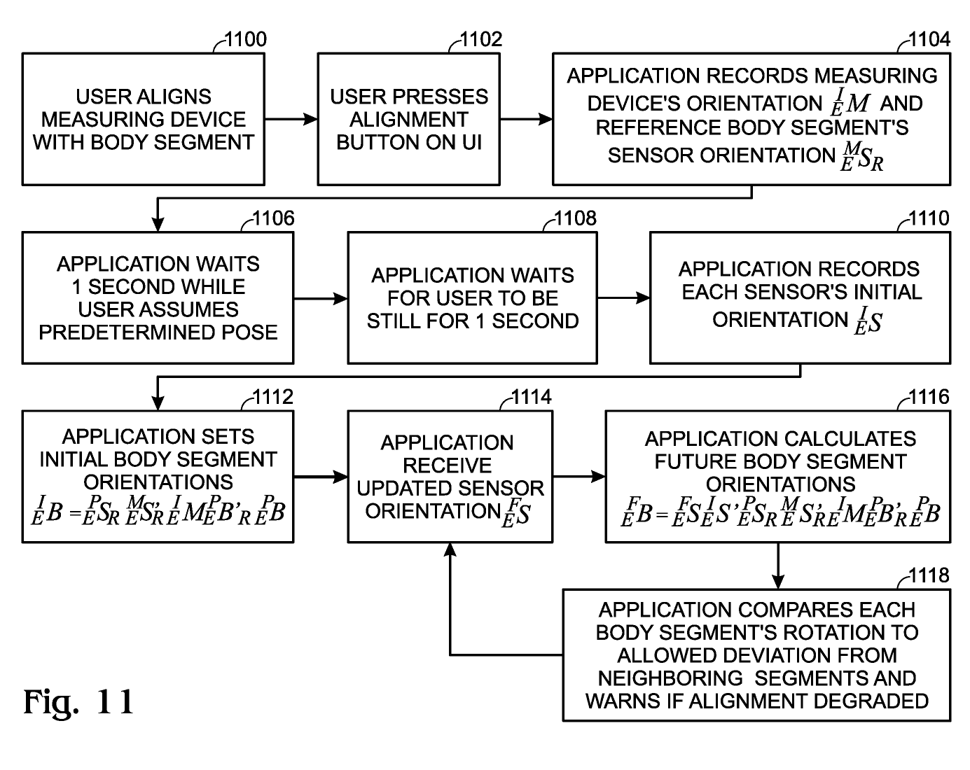

FIG. 11 is a flowchart illustrating a body segment orientation determination method based upon using a predetermined pose and an unknown direction with the aid of an EROMD. This method is similar to the “Predetermined pose, Predetermined direction” method of FIG. 10. However, the user has an Earth relative orientation measurement device (EROMD) which the user aligns with any body segment (Step 1100), and then presses a button when the device is aligned (Step 1102). The EROMD measures that body segment's orientation relative to Earth (Step 1104), and sends that reading to the application. The associated body segment's sensor reading is captured at the same time. The EROMD has alignment markings to aid in aligning it with the selected body segment. These markings may include lines or grids drawn on the device or projected by the device. The user then moves into the predetermined pose (Step 1106). After all the sensors are still (Step 1108), the application records the orientation of each sensor, along with the change in orientation of the reference segment sensor from when the EROMD measurement was made (Step 1110). These values are combined to determine the initial pose orientation and initial body segment sensor orientations (Step 1112). Sensor updates are received in Step 1114 and the body segment orientations are recalculated in Step 1116, using the initial orientations calculated in Step 1112.

The advantage of this alignment method is that it does not require the user to know a predetermined direction (such as south), which can be difficult without a compass. The user doesn't even need to know the direction of up, which means the user can purposely lie down instead of standing up while measuring alignment and without requiring a new pose to be added to the predetermined pose data base. Using a measuring device also has the benefit that someone other than the user can make the measurement, such as a health care provider.

More explicitly, the orientation of the measurement device, _{E}^{I}M, and the reference sensor. _{E}^{M}S_{R} are recorded in Step 1104 and Step 1110 records each sensor's initial orientation, _{E}^{I}S. The application sets each body segment's initial orientation equal to its pose orientation rotated by difference between the measured reference body segment's orientation _{E}^{I}M and its pose's inverse, _{E}^{P}B′_{R} in Step 1112, and further rotated by the change in the reference segment's sensor orientation between the time when the EROMD measurement occurred and when the pose measurement occurred:

_{E}^{I}B=_{E}^{I}S_{RE}^{M}S′_{RE}^{I}M_{E}^{P}B′_{RE}^{P}B

The future orientation of each body segment (Step 1116) is given by:

_{E}^{F}B=_{I}^{F}S_{E}^{I}B=_{E}^{F}S_{E}^{I}S′_{E}^{I}S_{RE}^{M}S′_{RE}^{I}M_{E}^{P}B′_{RE}^{P}B

Optionally, Step 1118 can compare future estimated body segment joint rotations to a musculoskeletal model and alert the user if the joint rotation is not within physiologically possible movements so that the user may perform the alignment process again.

### Known Pose Method 3: Predetermined Pose, Unknown Direction, Predetermined Move

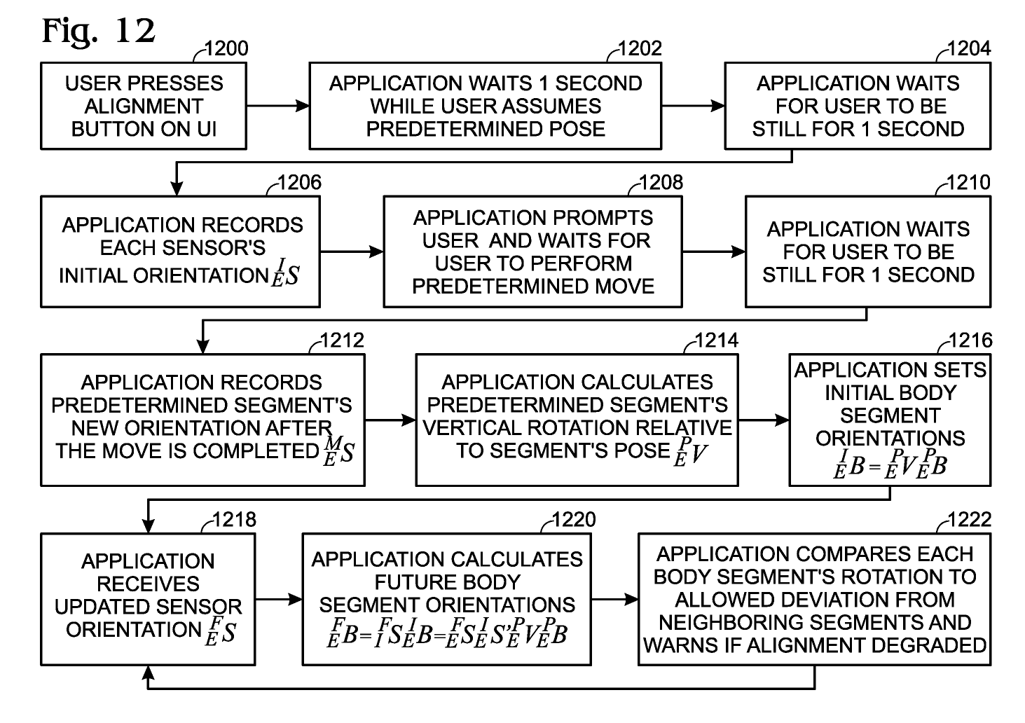

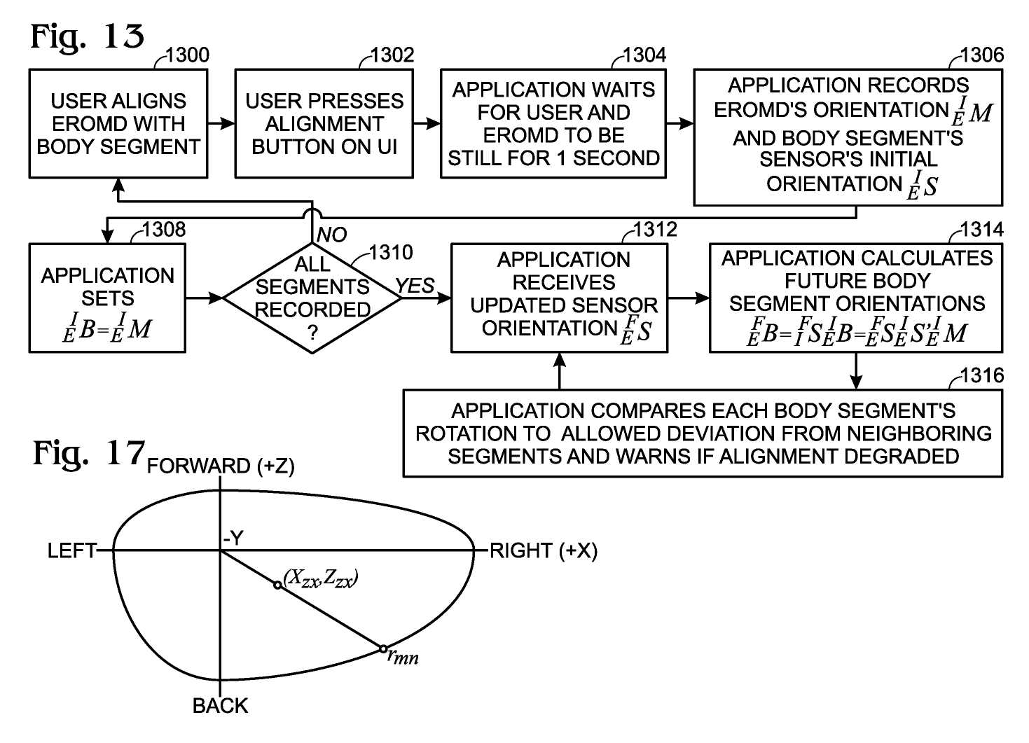

FIG. 12 is a flowchart illustrating a body segment orientation determination method based upon using a predetermined pose, facing an unknown direction, and making a predetermined move. This method is similar to the “Predetermined pose, predetermined direction” method of FIG. 10. However, after remaining still to record initial sensor orientations (Steps 1200 to 1206), the user makes a predetermined move recorded in Step 1212, to indicate which horizontal direction they are facing relative to Earth's surface. For example, they could point their arm forward, bend their knee, nod their head, lift their leg, etc. The method then calculates the orientation of the rotational axis (Step 1214) and from that orientation calculates the vertical rotation of the user, _{E}^{P}V.

While the predetermined move indicates the horizontal direction the user is facing, the method assumes the user knows and correctly aligns themselves in the vertical direction. Knowing the vertical direction is relatively easy, because it is just based on gravity, whose direction is easy for a human to detect.

To continue the example:

Up direction is determined by pose

Example: Standing means body segments are vertical

Example: Lying means body segments are horizontal

Forward direction is determined by well-defined movement

Example: Move arm from pointing down to pointing forward
Forward direction is perpendicular to rotational axis of predetermined move

Example: Move arm from pointing down to pointing to side
Forward direction is parallel or anti-parallel to axis of predetermined move
   The vertical rotation quaternion (_{E}^{P}V) is calculated as follows:
   Since the up direction is determined by the pose, only the south direction needs to be calculated:

\hat{j}=(0,1,0)=axial axis of body segment

bᵢ=(x_{bi},y_{bi},z_{bi})=_{E}^{I}Sĵ_{E}^{I}S′=initial orientation of body segment's axial axis

b_{f}=(x_{bf},y_{bf},z_{bf})=_{E}^{F}Sĵ_{E}^{F}S′=final orientation of body segment's axial axis

f=(x_{f},y_{f},0)=forward pointing vector

For the example where the user is standing and points their arm forward, the forward direction is given by the resultant vector of the initial orientation of the body segment's axial axis crossed with the final orientation of the body segment's axial axis crossed with a downward pointing unit vector.

f=bᵢxb_{f}x(0,0,−1)=(x_{bi}z_{bf}−z_{bi}x_{bf},y_{bi}z_{bf}−z_{bi}y_{bf},0)

For the example where the user is standing and points their right arm to the right side, the forward direction is given by the resultant vector of the final orientation of the body segment's axial axis crossed with the initial orientation of the body segment's axial axis multiplied by an identity vector with the Z-axis dimension zeroed.

f=b_{f}xbᵢ*(1,1,0)=(y_{bf}z_{bi}−z_{bf}y_{bi},x_{bf}z_{bi}−z_{bf}x_{bi},0)

For the example where the user is standing and points their right arm to the right side, the forward direction is given by the resultant vector of the initial orientation of the body segment's axial axis crossed with the final orientation of the body segment's axial axis multiplied by an identity vector with the Z-axis dimension zeroed.

f=bᵢxb_{f}*(1,1,0)=(y_{bi}z_{bf}−z_{bi}y_{bf},x_{bi}z_{bf}−z_{bi}x_{bf},0)

For any of these three examples, the forward pointing vector is normalized to a unit vector, \hat{f}.

$$\hat{f} = ( {x}_{fn} , {y}_{fn} , 0 ) = forward pointing unit vector$$

$$\hat{f} = ( {x}_{fn} , {y}_{fn} , 0 ) = \frac{f}{‖ f ‖} = \frac{f}{\sqrt{{x}_{f}^{2} + {y}_{f}^{2}}}$$

The vertical rotation quaternion, _{E}^{P}V, is then calculated using the half angle between the forward pointing unit vector, \hat{f}, and the Earth south vector, \hat{ι}.

_{E}^{P}V=(rᵥ,0,0,zᵥ)

ι=(1,0,0)=southward pointing vector

$$\begin{matrix}{r}_{v} = \frac{( 1 + {x}_{fn} )}{\sqrt{{( 1 + {x}_{fn} )}^{2} + {y}_{fn}^{2}}} \\ = \sqrt{\frac{{( 1 + {x}_{fn} )}^{2}}{{( 1 + {x}_{fn} )}^{2} + {y}_{fn}^{2}}} \\ = \sqrt{\frac{{( 1 + {x}_{fn} )}^{2}}{( 1 + 2 {x}_{fn} + {x}_{fn}^{2} ) + {y}_{fn}^{2}}} \\ = \sqrt{\frac{{( 1 + {x}_{fn} )}^{2}}{2 ( 1 + {x}_{fn} )}} \\ = \sqrt{\frac{( 1 + {x}_{fn} )}{2}}\end{matrix}$$

$$\begin{matrix}{z}_{v} = \frac{{y}_{fn}}{\sqrt{{( {x}_{fn} + 1 )}^{2} + {y}_{fn}^{2}}} \\ = sgn ( {y}_{f} ) \sqrt{\frac{{y}_{fn}^{2}}{{( 1 + {x}_{fn} )}^{2} + {y}_{fn}^{2}}} \\ = sgn ( {y}_{fn} ) \sqrt{\frac{{y}_{fn}^{2} + ( 1 - 1 )}{( 1 + 2 {x}_{fn} + {x}_{fn}^{2} ) + {y}_{fn}^{2}}} \\ = sgn ( {y}_{fn} ) \sqrt{\frac{{y}_{fn}^{2} + ( 1 - {x}_{fn}^{2} - {y}_{fn}^{2} )}{2 ( 1 + {x}_{fn} )}} \\ = sgn ( {y}_{fn} ) \sqrt{\frac{1 - {x}_{fn}^{2}}{2 ( 1 + {x}_{fn} )}} \\ = sgn ( {y}_{fn} ) \sqrt{\frac{( 1 + {x}_{fn} ) ( 1 - {x}_{fn} )}{2 ( 1 + {x}_{fn} )}} \\ = sgn ( {y}_{fn} ) \sqrt{\frac{( 1 - {x}_{fn} )}{2}}\end{matrix}$$

The advantage of this alignment method is that it does not require the user to know a predetermined direction or to use a measurement device. However, it does require that the user is able to align themselves in a vertical direction and be able to move, which may not be possible for a patient.

The application records the orientation (Step 1206) of the user after they are still in Step 1204, then prompts the user to perform a predetermined move and then remain still again (Steps 1208-1210). The method then records the orientation change of the move closest to 90 degrees (Step 1212) while the user remains still for the second time, as that is where the user is most accurate when pointing. The orientation of the body segment relative to Earth's surface is calculated in Step 1214. In Step 1216 each body segment's initial orientation is set equal to its pose orientation rotated by calculated vertical rotation:

_{E}^{I}B=_{E}^{P}V_{E}^{P}B

After updates in Step 1218, the future orientation of each body segment is given in Step 1220 by:

_{E}^{F}B=_{I}^{F}S_{E}^{I}B=_{E}^{F}S_{E}^{I}S′_{E}^{P}V_{E}^{P}B

Optionally, Step 1222 can compare future estimated body segment joint rotations to a musculoskeletal model and alert the user if the joint rotation is not within physiologically possible movements so that the user may perform the alignment process again.

### Arbitrary Pose

In addition to predetermined poses, body segment orientation can also be determined when the user is initially in an arbitrary pose. Two arbitrary pose methods described herein require estimating the initial orientation of each body segment separately, as opposed to predetermined poses where the initial orientation of each body segment was a known offset from the orientation of any other body segment, set by the predetermined pose. The advantages of these methods are that they do not require the user to be able to assume any particular pose, which might be required for medical patients with limited mobility. The disadvantage is that the initial pose needs to be either input by the use of secondary measuring devices, such as an EROMD or goniometer, or estimated based upon a musculoskeletal model of body segments combined with measured user movements.

### Arbitrary Pose Method 1: EROMD

FIG. 13 is a flowchart illustrating an arbitrary pose method for the determination of body segment orientation using an EROMD. The orientation of an arbitrarily posed body segment can be measured using an EROMD, such as the one described by FIG. 11 (Predetermined pose, unknown direction, Earth relative orientation measurement device). In this case, each body segment is measured separately and the body segment's orientation recorded by the application software.

_{E}^{I}B=_{E}^{I}M

The future orientation of each body segment is given by:

_{E}^{F}B=_{I}^{F}S_{E}^{I}B=_{E}^{F}S_{E}^{I}S′_{E}^{I}M

This method has the advantage of not requiring the user to be able to assume a predetermined pose or move, while still providing very accurate results. The disadvantage of this method is that the orientation of each body segment needs to be recorded separately.

The user aligns an EROMD with a body segment in (Step 1300), and then presses a button indicating device alignment is ready (Step 1302). After a pause in Step 1304, the EROMD orientation is measured and the associated body segment's sensor reading is captured at the same time (Step 1306). After setting the body segment's orientation to the EROMD's orientation (Step 1308) and repeating the alignment and orientation capture process for all body segments being tracked (Step 1310), updates are received in Step 1312 and subsequent (future) body segment orientation calculations are made in Step 1314. Optionally, Step 1316 can compare future estimated body segment joint rotations to a musculoskeletal model and alert the user if the joint rotation is not within physiologically possible movements so that the user may perform the alignment process again.

### Arbitrary Pose Method 2: Goniometer

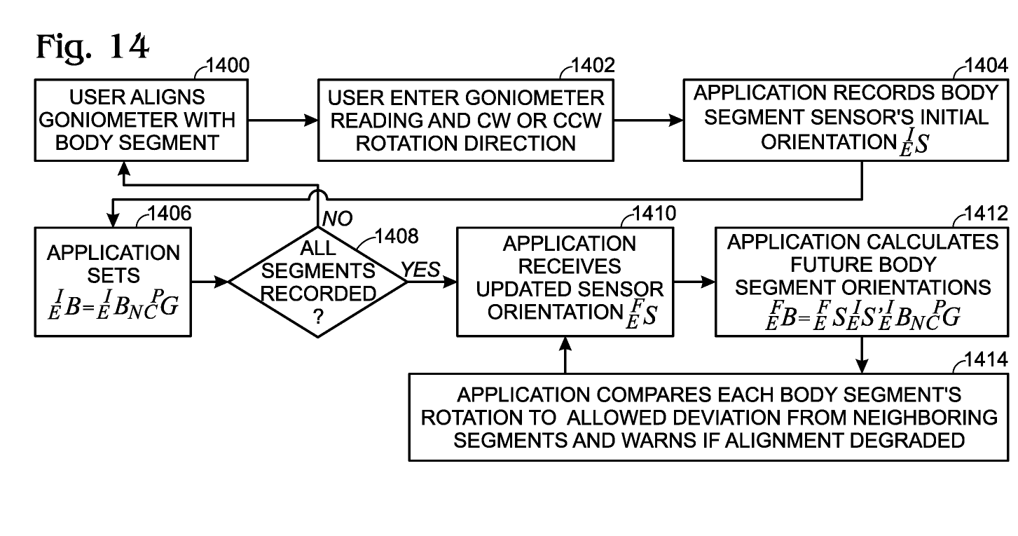

FIG. 14 is a flowchart illustrating an arbitrary pose method of determining body segment orientation using a goniometer. Instead of using the EROMD described above, a goniometer can also be used to calculate a current body segment's orientation relative to a neighbor body segment for adjoining body segments that only radially rotate on a single axis, such as the thigh and shank which are connected by the knee joint. The goniometer is aligned with the movement plane of the body segments and the angle θ between the current and neighbor body segments is read off and input to the application. The application must adjust the calculated orientation to compensate for that fact that goniometers report both positive and negative angles as positive, and that the user may have aligned the goniometer either parallel or anti-parallel to the current body segment's coordinate axis. These conditions can cause the sign of θ to reverse, which causes the sign of the (x, y, z) rotational axis to reverse. The sign of θ is determined by the following rules:
1. Reverse sign if current body segment is proximal of neighbor body segment;
2. Reverse sign if distal body segment is rotated clockwise relative to proximal for a rotation axis pointing outward from the user's right (X-axis), front (Y-axis), or top (Z-axis), depending upon the movement plane.

θ_{Q}=((current is proximal of neighbor)?−1:1)

*((distal rotates CW relative to proximal)?−1:1)θ_{G}

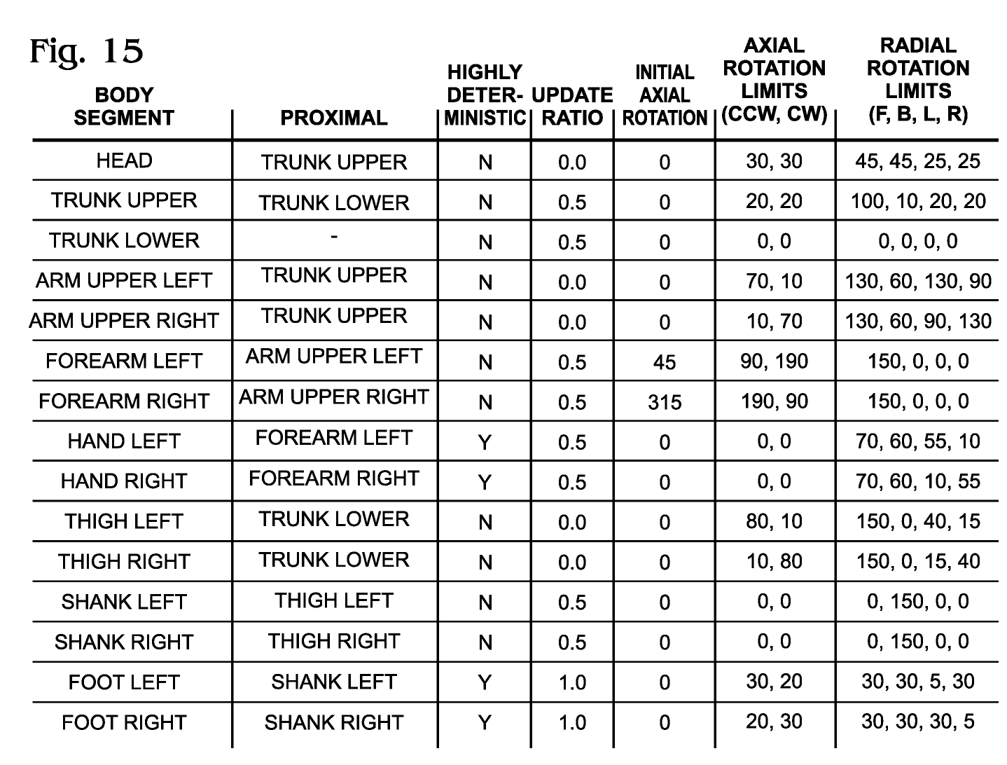

A proximal body segment is defined as the body segment next closest to the lower trunk from the current body segment. The table of FIG. 15 lists the proximal body segment for each body segment of relevance, along with additional information about each body segment, including its rotation relative to its proximal body segment.

The current body segment's orientation quaternion relative to the neighbor body segment is given by:

$${}_{D}^{P} G = goniometer quaternion ( proximal to distal )$$

$${}_{D}^{P} G = ( r , x , y , z )$$

$$r = cos ( \frac{{θ}_{Q}}{2} )$$

$$x , y , z = ( axis perpendicular to movement plane ? 1 \text{:} 0 ) \cdot sin ( \frac{{θ}_{Q}}{2} )$$

$${}_{E}^{I} B = {}_{E}^{I}{B}{}_{N}^{} {}_{D}^{P} G$$

$${}_{E}^{I}{B}{}_{N}^{} = neighbor limb quaternion ( {}_{E}^{I} B of neighbor limb )$$

The future orientation of each body segment is given by:

_{E}^{F}B=_{I}^{F}S_{E}^{I}B=_{E}^{F}S_{E}^{I}S′_{E}^{I}B_{ND}^{P}G

For orientation estimations relative to Earth, at least one body segment's orientation must be measured using an EROMD. Any combination of other body segments can be measured using either an EROMD or a goniometer. Using only a goniometer provides body segment orientations relative to each other, but not to Earth.

The user aligns the goniometer with a body segment (Step 1400), and enters the readings in Step 1402. The body sensor orientation is recorded in Step 1404, and set using the goniometer readings in Step 1406. After recording all the body segment initial orientations in Step 1408, future sensor updates received in Step 1410 are combined with the initial orientations to determine future body segment orientations in Step 1412. Optionally, Step 1414 can compare future estimated body segment joint rotations to a musculoskeletal model and alert the user if the joint rotation is not within physiologically possible movements so that the user may perform the alignment process again.

### Arbitrary Pose Method 3: Arbitrary Pose, Musculoskeletal Model

This method estimates the orientation of each body segment based upon a musculoskeletal model and without the use of any angular measurement device such as an EROMD or goniometer. The advantage of this method is in not requiring the user to assume any predetermined pose and not needing a secondary measurement device of any kind, such as an EROMD or goniometer. The disadvantage is that the quality of the estimate of the body segment orientations depends upon the range of joint rotations that a user has made. The estimate improves as the range increases and the motions approach the maximum limits of possible joint rotations allowed by the musculoskeletal model.

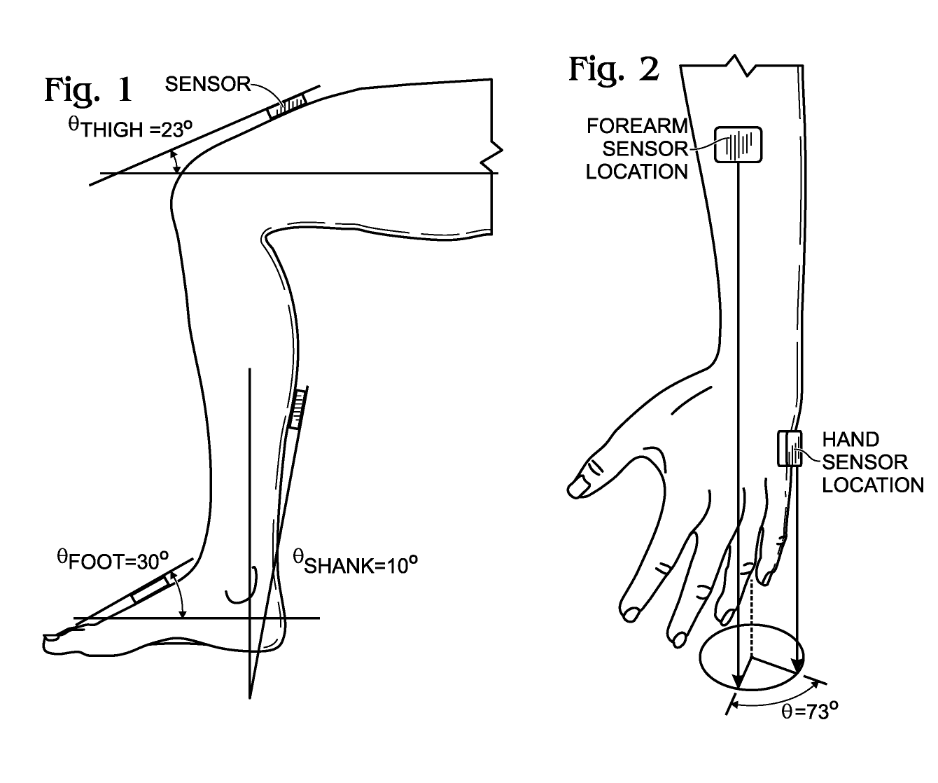

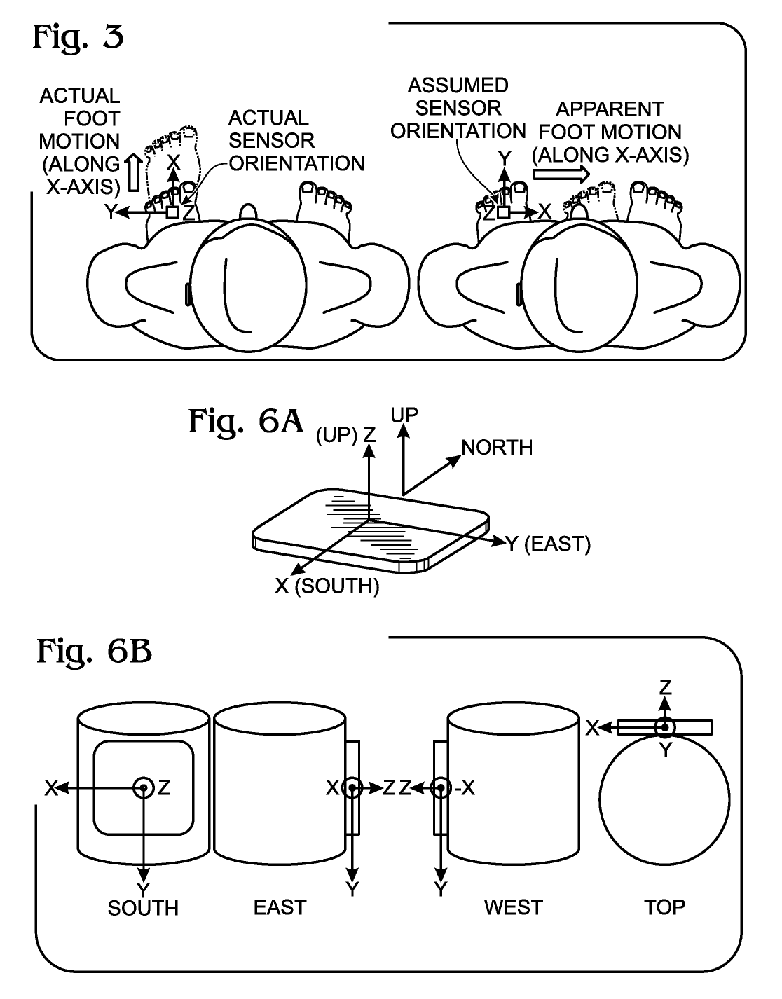

The best estimate for an arbitrary body segment orientation is derived when the sensors are placed on a body segment in a manner that aligns the sensors Y-axis with the body segment's axial axis, because then the radial axis offset is minimized. The problem of offsets is depicted in FIG. 1. Using the musculoskeletal model helps to reduce this offset, however, the user must approach the joint rotation limits of each body segment for the model to produce the most accurate results.

FIG. 15 is a table listing some exemplary parameters used in the musculoskeletal model, and exemplary values for body segments. Other values could be used for the physiological model based upon flexibility of user. For example, gymnast or back surgery patient.

The “Highly Deterministic” column indicates whether the sensor location on the body segment is constrained to a known location. This is true for the hands and feet, where, due to the shape of the body segment, there is only one location where the sensor could be placed. For the hand this is the back of the hand, and for the foot this is on the top. Knowing this location constrains the axial rotation of the sensor.

The “Update Ratio” column lists the fraction of sensor offset that is applied to the body segment when an offset estimate is calculated. The remainder of the offset is applied to its proximal body segment.

The “Initial Axial Rotation” column lists the assumed axial offset of a body segment from the axial orientation that body segment has in the predetermined standing pose shown in FIG. 7. Other predetermined poses could be used, such as sitting.

The “Axial Rotation Limits” column lists the clockwise and counter clockwise axial rotational limits of a body segment relative to its proximal segment in the predetermined standing pose shown in FIG. 7.

The “Radial Rotation Limits” column lists the forward, backward, left, and right radial rotational limits of a body segment relative to its proximal segment in the predetermined standing pose shown in FIG. 7.

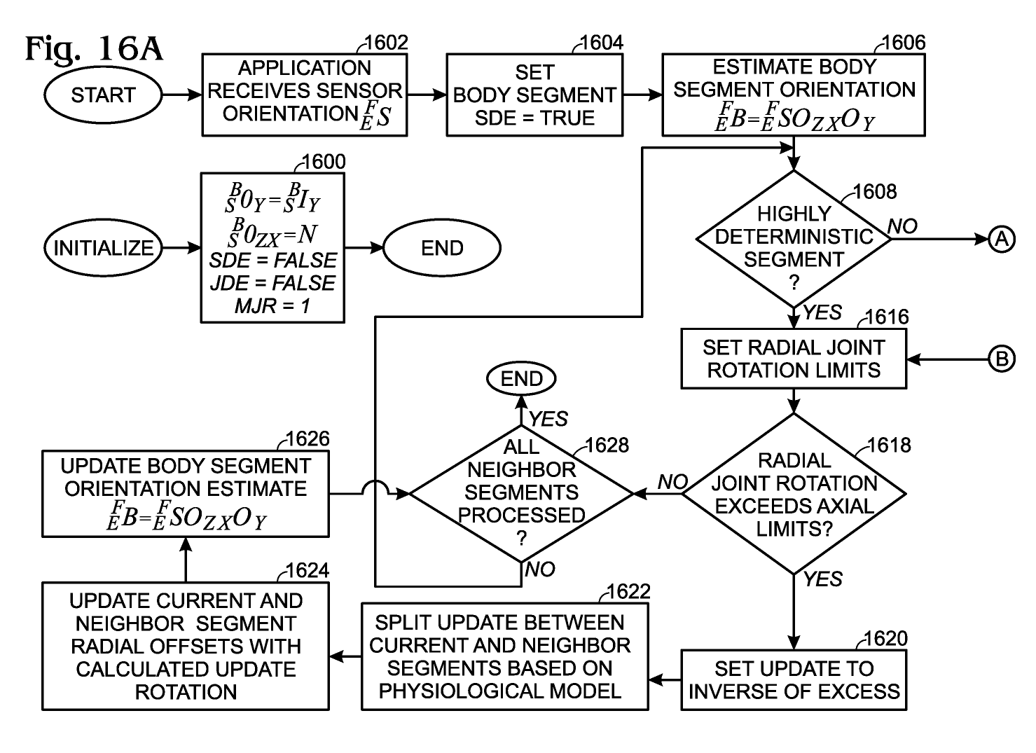

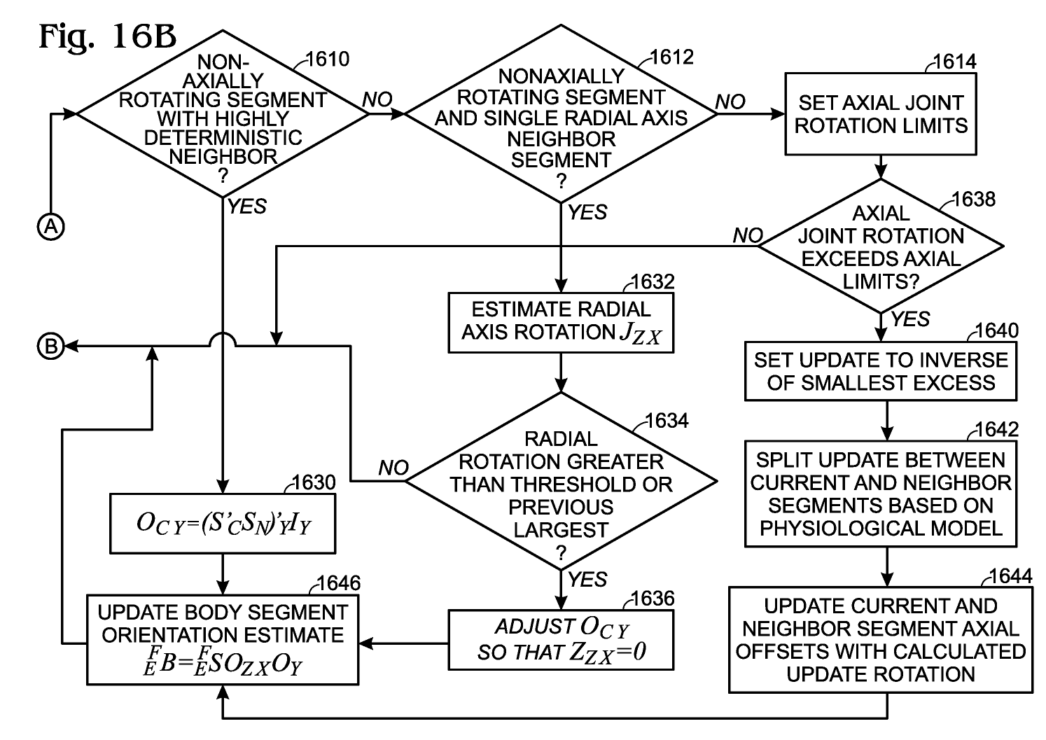

FIGS. 16A and 16B are a flowchart illustrating an arbitrary pose, musculoskeletal model for determining body segment orientation. When the system powers up or the user presses the alignment button on the application's UI, the application initializes itself by resetting all body segment radial offsets, O_{zx}, to the null quaternion value, N, and all axial offsets, O_{y}, to their initial axial rotation value, I_{y} (Step 1600). It also resets each segment's sensor data exists (SDE) and joint data exists (JDE) flags to false, and the minimum joint r_{zx} value (MJR) to 1. Step 1600 is performed once, at initialization.

Alternatively to initially setting an axial offset, O_{y}, to a predetermined value, I_{y}, the axial offset of a body segment can initially be set to the predetermined value, I_{y}, rotated by the initial joint axial rotation, J_{y}, between the body segment and its proximal neighbor body segment.

O_{y}=J_{y}I_{y}

The initial joint axial rotation, J_{y}, is determined by decomposing the initial joint composite rotation, J, into separate constituent axial and radial joint rotations (J_{zx},J_{y}) as described herein in the section titled “Joint rotation calculation”.

Each body segment sensor reports a value in Step 1602. This step occurs once for each sensor update, for example, at a rate of 20 Hertz times the number of sensors. In Step 1604 the SDE flag is set to true to indicate that data is available for the current sensor. In Step 1606 the segment's orientation is estimated from the sensor reading and the current axial and radial offsets.

_{E}^{F}B=_{E}^{F}SO_{zx}O_{y}

Next, the current body segment's type is checked to determine how to further process the sensor reading. Body segments are divided into 4 types, depending upon how well the sensor's position on the segment is known and how the segment moves relative to its neighbor segments. The first type (Step 1608) is highly deterministic segments (HD), described earlier. The second type (Step 1610) is segments that do not axially rotate relative to a highly deterministic neighbor segment (NA_HDN). The forearms are the only example of this type of segment, as the neighboring hands are highly deterministic segments which do not axially rotate relative to the forearm. The third type (Step 1612) is segments which only rotate on a single radial axis (SRA), such as the forearm relative to the upper arm, or the shank relative to the thigh. The fourth type (Step 1614) is segments which rotate on a multiple radial axes (MRA), such as the head relative to the upper trunk, or the lower trunk relative to the upper trunk or thighs. Segment types can be identified using the table on FIG. 15 based upon the “Highly Deterministic” column and the values in the axial and radial rotational limits columns.

Joint rotational limits (Step 1616) are used to estimate the orientation of the sensor on the body segment as the user moves. The relative orientations between the current body segment and its neighbor body segments are calculated for neighbors that have a sensor reading, i.e., neighbors that have their SDE flag set to true. If an estimated rotation of a joint exceeds a limit (Step 1618), then the sensor offset is updated to bring the estimated joint rotation in compliance with the musculoskeletal model. Otherwise, the method proceeds to Step 1628 to process additional neighboring body segments.

The joint constituent rotations (J_{zx}, J_{y}) between a current and a neighbor segment are calculated as described earlier herein in the section titled “Joint rotation calculation”.

The joint radial and axial rotation components (J_{zx}, J_{y}) are compared against the joint rotation limits, and if the rotational components exceed those limits, then the sensor orientation offset estimates are updated as exceeding these limits indicates that current offset values are likely not correct. First the axial offset is updated, after which the body segment orientation and joint rotations are recomputed, and then the radial offset is updated (Steps 1624 and 1626).

For highly deterministic segments (HD), the axial offset always remains at the initial axial rotation, I_{y}, so no update is required.

For non-axially rotating segments with a highly deterministic neighbor (NA_HDN), the axial offset between the current segment and the highly deterministic neighbor segment is kept to the null rotation, N, Step 1630. As shown below, the axial offset is then just set to the axial offset between the current and neighboring segment sensors.

J_{y}=N

(B′_{N}B_{C})_{y}=N

((S_{N}O_{Nzx}O_{Ny})′(S_{C}O_{Czx}O_{Cy}))_{y}=((O′_{Ny}O′_{Nzx}S′_{N})(S_{C}O_{Czx}O_{Cy})_{y}=N

O_{Ny}=I_{Ny} because highly deterministic neighbor segment

(O′_{Nzx}I′_{Ny}S′_{N}S_{C}O_{Czx}O_{Cy})_{y}=I′_{Ny}(S′_{N}S_{C}O_{Czx})_{y}O_{Cy}=I′N_{y}(S′_{N}S_{C})_{y}O_{Cy}=N

O_{Cy}=(S′_{N}S_{C})′I_{Ny}

For segments which do not axially rotate and have a neighbor segment that only rotates on a single radial axis (SRA), the axial offset between current segment and the neighboring segment is set such that the current segment's major radial axis is aligned with its X-axis. The major radial axis for an SRA segment is the only radial axis that the segment rotates on (Step 1632).

J_{zx}=(r_{zx},x_{zx},0,0) when X-axis is aligned with current segment's major radial axis

The equations below show how to calculate the axial orientation offset update to align the X-axis with the major radial axis. The direction of the rotational update axis is dependent upon the Z-axis sign of the joint's rotational axis, whether the current segment is a distal of the neighbor segment, and whether the distal segment of the current and neighbor segment pair rotates clockwise.

J_{zx}=(B′_{N}B_{C})_{zx}=(B′_{N}S_{C}O_{Czx}O_{Cy})_{zx}=(r_{zx},x_{zx},0,z_{zx})

The greatest rotation axis orientation accuracy is obtained when the joint rotation is closest to 90 degrees. However, the sensor may also inadvertently move over time, so the axial offset is updated whenever the current radial angle is larger than the maximum previous radial angle, or larger than a predetermined value (Step 1634), for example 45 degrees.

$$Update {O}_{Cy} , \\ if ( {r}_{zx , current} < {r}_{zx , previous\_max} ) or ( {r}_{zx , current} < cos ( \frac{π / 4}{2} ) )$$

The axial offset update is calculated and applied in Step 1636 as follows:

$$Adjust {O}_{Cy} so that {z}_{zx} = 0$$

$${O}_{Cy , new} = {O}_{Cy , previous} {O}_{Cy , update}$$

$${O}_{Cy , update} = ( {r}_{u} , 0 , {y}_{u} , 0 )$$

$${r}_{u} = \sqrt{\frac{( 1 + {x}_{n} )}{2}}$$

$${y}_{u} = {s}_{y} \sqrt{\frac{( 1 - {x}_{n} )}{2}}$$

$${x}_{n} = \frac{{s}_{x} {x}_{zx}}{\sqrt{{x}_{zx}^{2} + {z}_{zx}^{2}}}$$

$${s}_{x} = ( ( current == distal ) ? - 1 \text{:} 1 ) ( ( distal rotates clockwise ) ? - 1 \text{:} 1 )$$

$${s}_{y} = ( ( {z}_{zx} < 0 ) ? 1 \text{:} - 1 ) ( ( current == distal ) ? - 1 \text{:} 1 ) \\ * ( ( distal rotates clockwise ) ? - 1 \text{:} 1 )$$

When the current body segment has a neighboring distal body segment, the distal body segment's axial offset is also updated with the same axial update value, O_{Cy,update}.

Optionally, to accommodate joints which might have some slippage on the Z-axis, such as a loose knee joint, the axial offset value can be set to the mid-point of the maximum and minimum axial offsets calculated over time.

$${O}_{y} = \frac{PosDef ( y , {O}_{y , maximum} ) + PosDef ( y , {O}_{y , minimum} )}{2}$$

For segments which rotate on multiple radial axes (MRA), the axial offset between the current segment and each neighboring segment is set such that the rotation does not exceed a predetermined limit in both the clockwise and counterclockwise rotational directions. The limit is based upon a physiological model of the joint and examples are listed in FIG. 15.

$${J}_{limit} = ( {r}_{lm} , 0 , {y}_{lm} , 0 )$$

$${r}_{lm} = ( ( direction == clockwise ) ? - 1 \text{:} 1 ) cos ( \frac{{θ}_{limit}}{2} )$$

$${y}_{lm} = sin ( \frac{{θ}_{limit}}{2} )$$

The decomposed joint's positive definite Y-axis rotation is compared to the joint's clockwise and counterclockwise limits. If the rotation is within these limits (Step 1638), then the axial offset is not updated.

Jₜₑₛₜ=(rₜ,0,yₜ,0)=PosDef(y,J_{y})

If (rₜ≧r_{lm,CCW}) or (rₜ≦r_{lm,CW}) then don't update O_{y}

If the joint is outside the limits, then the axial offset is updated so that the joint rotation resides inside the closest limit. The closest limit is calculated by rotating the joint by each limit and choosing the rotation with the smallest angular excess (Step 1640). The MRA axial update is equal to the inverse of the excess rotation.

J_{excess,CCW}=PosDef(y,J_{y}J′_{limit,CCW})

J_{excess,CW}=NegDef(y,J_{y}J′_{limit,CW})

If (r_{excess,CCW}>r_{excess,CW})

then O_{y,update,}=J′_{excess,CCW}

else O_{y,update}=J′_{excess,CW}

If the current segment is a proximal segment, then the update value is inverted, otherwise, it is not.

If (current==proximal)

then O_{y,update,current}=O′_{y,update}

else O_{y,update,current}=O_{y,update}

The neighbor segment's update is the inverse of the current segment's update.

O_{y,update,neighbor}=O_{y,update,current}

The axial update is split between the current and neighbor segments based upon a physiological model (Step 1642). Example update ratios are listed in the table of FIG. 15 for current segments which are distal segments.

ratioValue_{current}=(current==distal)?tableRatioValue:(1−tableRatioValue)

The neighbor ratio value is equal to one minus the current ratio value.

ratioValue_{neighbor}=1−ratioValue_{current}

The axial offset update value is mixed with the null rotation using the ratio value to form a ratio rotation, and then that ratio rotation is then used to update the existing axial offset (Step 1644). Each element of the axial update is multiplied by the ratio value and then summed with a null rotation which had its elements multiplied by one minus the ratio value.

O_{y,ratio}=(ratioValue O_{y,update})+((1−ratioValue)N)

O_{y,new}=O_{y,existing}O_{y,ratio}

The current and neighbor body segment orientations are updated after the axial orientation offsets are updated (Step 1646).

FIG. 17 is a continuous elliptical model of joint rotation. Similar to the axial offset updating method of an MRA segment, each body segment's radial offset is updated based upon radial rotational limits with its neighboring body segments. Radial rotation limits are defined for the forward, back, left, and right directions of each segment. The table of FIG. 15 lists example radial rotation limits. Off axis limits are calculated by determining the radial rotation axis quadrant from the signs of joint's x and z decomposed rotation elements, then using the following equation based on a piecewise continuous elliptical model of joint rotation (see FIG. 16, Step 1616).

$${J}_{limit} = ( {r}_{lm} , {x}_{lm} , 0 , {z}_{lm} )$$

$${r}_{lm} = {r}_{mm}$$

$${x}_{lm} = {x}_{zx} \sqrt{\frac{1 - {r}_{lm}^{2}}{{x}_{zx}^{2} + {z}_{zx}^{2}}}$$

$${z}_{lm} = {z}_{zx} \sqrt{\frac{1 - {r}_{lm}^{2}}{{x}_{zx}^{2} + {z}_{zx}^{2}}}$$

$${r}_{mn} = {r}_{x} {r}_{z} \sqrt{\frac{( {x}_{zx}^{2} + {z}_{zx}^{2} )}{{r}_{z}^{2} {x}_{zx}^{2} + {r}_{x}^{2} {z}_{zx}^{2}}}$$

$${r}_{x} = cos ( \frac{{θ}_{limit , x}}{2} ) = X - axis limit ( front \& back )$$

$${r}_{z} = cos ( \frac{{θ}_{limit , z}}{2} ) = Z - axis limit ( left \& right )$$

The radial offset needs to be updated if the joint's rotational angle exceeds the rotational limits (Step 1618).

J_{zx}=(r_{zx},x_{zx},0,z_{zx})

J_{zx}=PosDef(r,((current==proximal)?J:J′)_{zx})

If (r_{zx}<r₁) then update O_{zx}

The radial offset is updated similar to the method used for the axial rotation offset update. The update is set to the inverse of the excess rotation (Step 1620).

J_{excess}=J_{zx}J′ₗᵢₘᵢₜ

O_{zx,update}=J′_{excess}

If the current segment is a proximal segment, then the update value is inverted, otherwise, it is not.

If (current==proximal)

then O_{zx,update,current}=O′_{zx,update}

else O_{zx,update,current}=O_{zx,update}

The neighbor segment's update is the inverse of the current segment's update.

O_{zx,update,neighbor}=O_{zx,update,current}

The radial update is split between the current and neighbor segments based upon a physiological model (Step 1622).

O_{zx,ratio}=(ratioValue O_{zx,update})+((1−ratioValue)N)

O_{zx,new}=O_{zx,existing}O_{zx,ratio}

The current and neighbor body segment orientations radial offsets are updated (Step 1626), and then the body segment orientations are updated (Step 1626).

Joint limits are checked for the remaining neighboring segments (Step 1628).

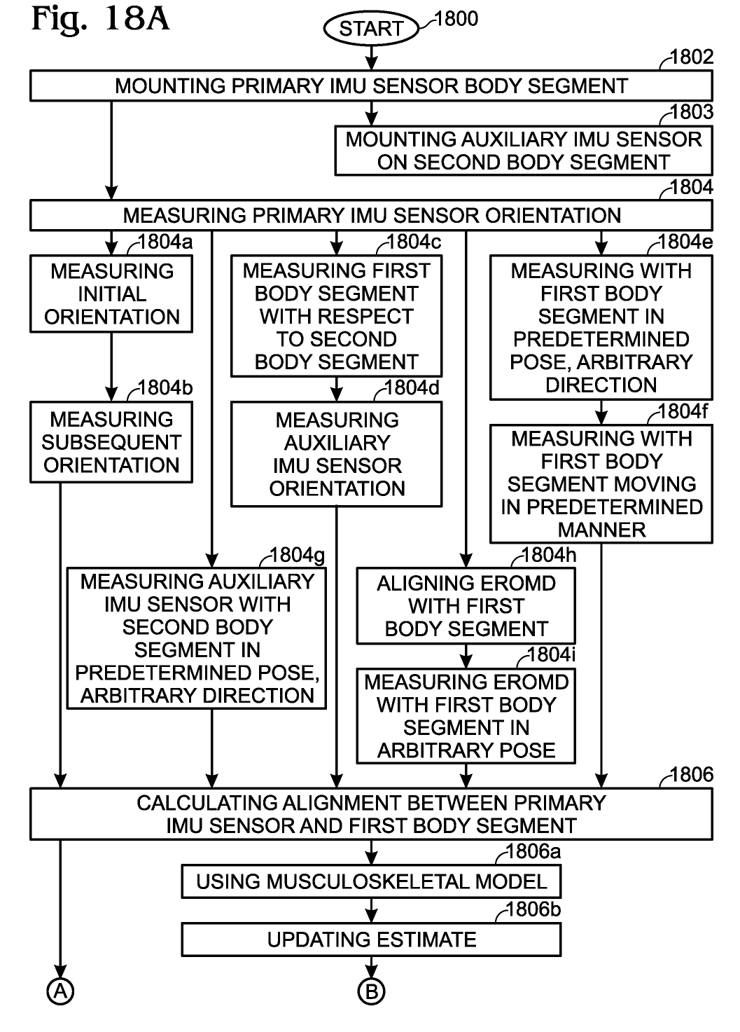

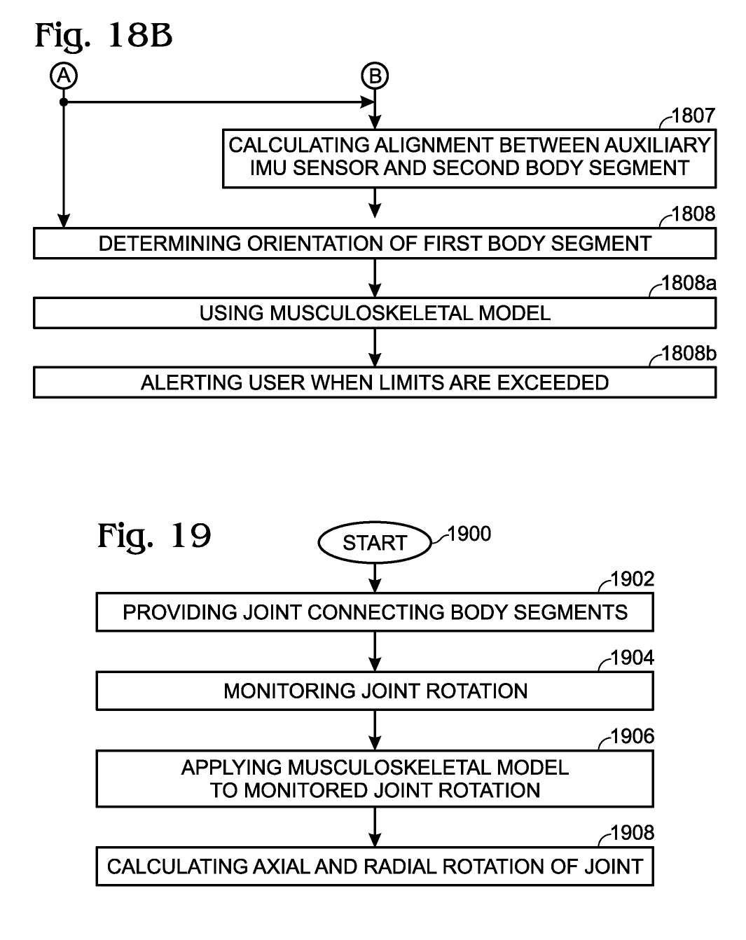

FIGS. 18A and 18B are a flowchart summarizing the above-described method for determining the orientation of a body segment using an IMU sensor capable of measuring its orientation relative to Earth. The method begins at Step 1800. Step 1802 mounts a primary IMU sensor on a first body segment, with an unknown first alignment orientation relationship between the primary IMU sensor and the first body segment. Step 1804 measures the primary IMU sensor orientation. In one aspect, Step 1804 measures the primary IMU sensor orientation with the first body segment in a predetermined pose, aligned in a predetermined direction relative to Earth. Step 1806 calculates the alignment orientation relationship between the primary IMU sensor orientation and the first body segment orientation. In one aspect, Step 1804a measures a primary IMU sensor initial orientation, and Step 1804b measures a subsequent orientation. In response to the primary IMU sensor initial and subsequent orientations (Steps 1804a and 1804b), and calculating the alignment orientation relationship between the primary IMU sensor initial orientation and the first body segment orientation (Step 1806), Step 1808 determines a subsequent orientation of the first body segment.

In one aspect, determining the subsequent orientation of first body segment in Step 1808 comprises the following substeps. Step 1808a uses a body segment musculoskeletal model describing potential movement relationships between adjacent body segments to find deterministic, axial rotation, and radial rotation limits associated with the first body segment. Deterministic limits describe the likely accuracy of a first alignment orientation relationship estimate. Step 1808b alerts a user when the estimated relationship between adjacent body segments exceeds the limits of the musculoskeletal model.

In a different aspect, Step 1804c measures the first body segment orientation with respect to a second body segment using a goniometer. Simultaneous with measuring the primary IMU sensor orientation, Step 1804d measures the orientation of an auxiliary IMU sensor mounted on the second body segment, where an alignment orientation relationship between the auxiliary IMU sensor and second body segment is known.

In another aspect, Step 1804e measures the primary IMU sensor first orientation with the first body segment in a predetermined pose, aligned in an arbitrary direction relative to Earth. Step 1804f measures the primary IMU sensor second orientation with the first body segment moving in a predetermined manner.

In another variation, Step 1803 mounts an auxiliary IMU sensor on a second body segment with an unknown second alignment orientation relationship between the auxiliary IMU sensor and second body segment. In this variation the second body segment is in a predetermined pose, aligned in an arbitrary direction relative to Earth. Simultaneous with measuring the primary IMU sensor's first orientation in Step 1804e, Step 1804g measures the auxiliary IMU sensor orientation. Step 1807 calculates an auxiliary alignment orientation relationship between the auxiliary IMU sensor orientation and the second body segment orientation.

In another aspect, Step 1804h aligns an EROMD with the first body segment. Simultaneous with measuring the primary IMU sensor orientation, Step 1804i measures the EROMD orientation with the first body segment in an arbitrary pose, in an arbitrary direction relative to Earth.

In one aspect, measuring the primary IMU sensor orientation in Step 1804 includes the following substeps. Step 1804h aligns an EROMD with a predetermined second body segment. Simultaneous with measuring the primary IMU sensor orientation, Step 1804i measures the EROMD orientation with the second body segment in a predetermined pose, in an arbitrary direction relative to Earth.

In yet another aspect, Step 1804 estimates the alignment orientation relationship between the primary IMU sensor orientation and the first body segment orientation. The calculation of the alignment orientation relationship between the primary IMU sensor orientation and the first body segment orientation in Step 1806 includes the following substeps. Step 1806a uses a body segment musculoskeletal model describing potential movement relationships between adjacent body segments to find deterministic, axial rotation, and radial rotation limits associated with the first body segment. In response to comparing the deterministic, axial rotation, and radial rotation limits with subsequent calculated movement relationships between the first body segment and an adjacent body segment, Step 1806b updates the estimated alignment orientation relationship.

### Joint Rotation Calculation

The joint rotation between a current and a neighbor segment can be calculated by the following formulas.

B_{C}=B_{NN}^{C}J=current segment orientation

B_{N}=neighbor segment orientation

_{N}^{C}J=joint rotation between current and neighbor segments

_{N}^{C}J=B′_{N}B_{C}

Joint axial and radial rotation values are determined by decomposing the composite joint rotation into separate constituent axial and radial joint rotations, applying a physiological model during the decomposition to obtain joint rotations which are physiologically possible.

Physiologically, only radial rotations on the X-axis can be obtuse, for example, the knee and elbow joints. Z-axis rotations are always acute. To model this, a composite XYZ rotation is first decomposed into two possible decomposed rotation sets, each set including one axial and one radial rotation. The first set is based upon the original composite rotation. The second set is based upon a rotated composite rotation equal to the original composite rotation rotated by 180 degrees on the X-axis, with the results then rotated back 180 degrees to the original orientation. The two sets are then mixed together using their positive definite values to obtain a final decomposed rotation set. The dimension (r, x, y, or z) used for positive definite processing is the dimension which contains the largest valued vector elements, determined by comparing the sum of the absolute values of the vector elements for each dimension. The mixing factor is based upon the projection of a unit Y-axis vector rotated by the original rotation onto the Y-axis. As shown below, the two sets are defined and mixed, along with the calculation of the Y-axis unit vector projection and the mixing factor.

$${Q}_{0} = {Q}_{xyz} = ( r , x , y , z ) = original composite rotation \\ ( {Q}_{zx} , {Q}_{y} ) = final decomposed rotation set$$

$$\begin{matrix}{( {Q}_{zx} , {Q}_{y} )}_{0} = ( {Q}_{0 zx} , {Q}_{0 y} ) \\ = ( ( {r}_{0 zx} , {x}_{0 zx} , 0 , {z}_{0 zx} ) , ( {r}_{0 y} , 0 , {y}_{0 y} , 0 ) ) \\ = non \text{-} rotated decomposed rotation set\end{matrix}$$

$$\begin{matrix}{( {Q}_{zx} , {Q}_{y} )}_{{n}^{2}} = ( {Q}_{{π}^{2} zx} , {Q}_{{π}^{2} y} ) \\ = ( ( {r}_{{π}^{2} zx} , {x}_{{π}^{2} zx} , 0 , {z}_{{π}^{2} zx} ) , ( {r}_{{π}^{2} y} , 0 , {y}_{{π}^{2} y} , 0 ) ) \\ = 180 degree double rotated decomposed rotation set\end{matrix}$$

$${Q}_{zx} = mPosDef ( {L}_{zx} , {Q}_{0 zx} ) + ( 1 - m ) PosDef ( {l}_{zx} , {Q}_{{π}^{2} zx} )$$

$${Q}_{y} = mPosDef ( {L}_{y} , {Q}_{0 y} ) + ( 1 - m ) PosDef ( {l}_{y} , {Q}_{{π}^{2} y} )$$

$$m = \frac{1}{1 + {e}^{- α ( {p}_{y} + \sqrt{2} )}} = mixing factor$$

$$α = 10 = mixing gain$$

$${l}_{zx} = dimension of largest elements in {Q}_{0 zx} and {Q}_{{π}^{2} zx}$$

$${l}_{y} = dimension of largest elements in {Q}_{0 y} and {Q}_{{π}^{2} y}$$

$${p}_{y} = \hat{J} \cdot ( {Q}_{xyz} \hat{J} {Q}_{xyz}^{′} ) = y unit vector projection$$

$$\hat{J} = ( 0 , 1 , 0 ) = y unit vector$$

The first set of decomposed rotations is calculated as follows:

$${Q}_{0} = {Q}_{0 zx} {Q}_{0 y}$$

$${Q}_{0 zx} {Q}_{0 y} = ( {r}_{0 zx} , {x}_{0 zx} , 0 , {z}_{0 zx} ) ( {r}_{0 y} , 0 , {y}_{0 y} , 0 )$$

$$If ( y == 0 )$$

$$Then$$

$${Q}_{0 y} = ( 1 , 0 , 0 , 0 )$$

$${Q}_{0 zx} = ( r , x , 0 , z )$$

$$Else$$

$${r}_{0 zx} = \sqrt{{r}^{2} + {y}^{2}}$$

$${x}_{0 zx} = \frac{rx + yz}{\sqrt{{r}^{2} + {y}^{2}}}$$

$${z}_{0 zx} = \frac{rz + xy}{\sqrt{{r}^{2} + {y}^{2}}}$$

$${r}_{0 y} = \frac{r}{\sqrt{{r}^{2} + {y}^{2}}}$$

$${y}_{0 y} = \frac{y}{\sqrt{{r}^{2} + {y}^{2}}}$$

The second set of decomposed rotations is calculated similar to the first set, except that the original composite rotation is first rotated 180 degrees along the X-axis to Q_{π}, then Q_{π} is decomposed and mapped to rotations originating at 0 degrees with reduced Z-axis contributions to generate Q_{π}_{²}.

$${Q}_{π} = ( {r}_{π} , {x}_{π} , {y}_{π} , {z}_{π} ) = {Q}_{\hat{i}} {Q}_{0}$$

$${Q}_{\hat{i}} = ( 0 , 1 , 0 , 0 )$$

$${Q}_{π} = ( x , - r , z , - y )$$

$$If ( {y}_{π} == 0 ) i . e . if ( z == 0 )$$

$$Then$$

$${Q}_{π y} = ( 1 , 0 , 0 , 0 )$$

$${Q}_{π zx} = ( {r}_{π} , {x}_{π} , 0 , {z}_{π} ) = ( x , - r , 0 , - y )$$

$$Else$$

$${r}_{π zx} = \sqrt{{r}_{π}^{2} + {y}_{π}^{2}} = \sqrt{{x}^{2} + {z}^{2}}$$

$${x}_{π zx} = \frac{{r}_{π} {x}_{π} + {y}_{π} {z}_{π}}{\sqrt{{r}_{π}^{2} + {y}_{π}^{2}}} = \frac{- rx - yz}{\sqrt{{x}^{2} + {z}^{2}}}$$

$${z}_{π zx} = \frac{{r}_{π} {z}_{π} - {x}_{π} {y}_{π}}{\sqrt{{r}_{π}^{2} + {y}_{π}^{2}}} = \frac{- xy + rz}{\sqrt{{x}^{2} + {z}^{2}}}$$

$${r}_{π y} = \frac{{r}_{π}}{\sqrt{{r}_{π}^{2} + {y}_{π}^{2}}} = \frac{x}{\sqrt{{x}^{2} + {z}^{2}}}$$

$${y}_{π y} = \frac{{y}_{π}}{\sqrt{{r}_{π}^{2} + {y}_{π}^{2}}} = \frac{z}{\sqrt{{x}^{2} + {z}^{2}}}$$

The rotated radial decomposition then has its Z-axis element reduced, based upon the Y-axis unit vector projection p_{y} calculated earlier.

$${z}_{{π}_{δ} zx} = δ {z}_{π zx}$$

$$δ = \frac{1}{1 + {e}^{- β {p}_{y}}} = reduction factor$$

$$β = 10 = reduction gain$$

The remaining elements in Q_{πzx} are scaled to create a normalized Q_{πδzx}.

$${r}_{{π}_{δ} zx} = {nr}_{π zx}$$

$${x}_{{π}_{δ} zx} = {nx}_{π zx}$$

$$n = \sqrt{\frac{1 - {z}_{{π}_{δ} zx}^{2}}{{r}_{π zx}^{2} + {x}_{π zx}^{2}}}$$

Q_{πδzx} is then mapped to a rotation from 0 degrees to create Q_{π}_{²}_{zx}.

If (abs(r_{πδzx})>abs(z_{πδzx})) then

r_{π}_{²}_{zx}=\sqrt{1−r_{π}²_{δzx}}

x_{π}_{²}_{zx}=−sgn(x_{πδzx})\sqrt{r_{π}²_{δzx}−z_{π}²_{δzx}}

y_{π}_{²}_{zx}=0

z_{π}²_{zx}=Z_{πδzx}

Else

Q_{π}²_{zx}=N

The rotated axial decomposition is mapped directly to complete axial the X-axis biased decomposition.

Q_{π}_{²}_{y}=(r_{πy},0,y_{πy},0)

FIG. 19 is a flowchart illustrating a method for determining separate constituent axial and radial rotations of a connected joint. The method begins at Step 1900. Step 1902 provides a joint connecting two adjoining body segments, a distal body segment connected to a proximal body segment. Step 1904 monitors (i.e., measures with an IMU) a composite joint rotation. Step 1906 applies a musculoskeletal model of the joint to the monitored joint rotation, where the model permits only decompositions with physiologically possible constituent rotations. Step 1908 calculates axial and radial rotations of the distal body segment relative to the proximal body segment.

A system and method have been provided for using one or more IMU sensors to determine the orientation of body segments. Examples of particular algorithms and hardware units have been presented to illustrate the invention. However, the invention is not limited to merely these examples. Other variations and embodiments of the invention will occur to those skilled in the art.

We claim:

1. A method for determining the orientation of a body segment using an inertial measurement unit (IMU) sensor capable of measuring its orientation relative to Earth, the method comprising:
mounting a primary IMU sensor on a first body segment, with an unknown first alignment orientation relationship between the primary IMU sensor and the first body segment;
measuring a primary IMU sensor orientation;
calculating an alignment orientation relationship between the primary IMU sensor orientation and a first body segment orientation.

2. The method of claim 1 further comprising:
measuring a primary IMU sensor initial orientation and a subsequent orientation; and,
in response to the primary IMU sensor initial and subsequent orientations, and calculating the alignment orientation relationship between the primary IMU sensor initial orientation and the first body segment orientation, determining a subsequent orientation of the first body segment.

3. The method of claim 2 wherein the determining the subsequent orientation of first body segment comprises:
using a body segment musculoskeletal model describing potential movement relationships between adjacent body segments to find deterministic, axial rotation, and radial rotation limits associated with the first body segment, where deterministic limits describe the likely accuracy of a first alignment orientation relationship estimate; and,
alerting a user when the estimated relationship between adjacent body segments exceeds the limits of the musculoskeletal model.

4. The method of claim 1 wherein measuring the primary IMU sensor orientation includes measuring the primary IMU sensor orientation with the first body segment in a predetermined pose, aligned in a predetermined direction relative to Earth.

5. The method of claim 1 wherein measuring the primary IMU sensor orientation includes:
aligning an Earth relative orientation measurement device (EROMD) with a predetermined second body segment;
simultaneous with measuring the primary IMU sensor orientation, measuring an EROMD orientation with the second body segment in a predetermined pose, in an arbitrary direction relative to Earth.

6. The method of claim 1 wherein measuring the primary IMU sensor orientation includes:
measuring the first body segment orientation with respect to a second body segment using a goniometer; and,
simultaneous with measuring the primary IMU sensor orientation, measuring an orientation of an auxiliary IMU sensor mounted on the second body segment, where an alignment orientation relationship between the auxiliary IMU sensor and second body segment is known.

7. The method of claim 1 wherein measuring the primary IMU sensor orientation includes:
measuring a primary IMU sensor first orientation with the first body segment in a predetermined pose, aligned in an arbitrary direction relative to Earth; and,
measuring a primary IMU sensor second orientation with the first body segment moving in a predetermined manner.

8. The method of claim 7 further comprising:
mounting an auxiliary IMU sensor on a second body segment with an unknown second alignment orientation relationship between the auxiliary IMU sensor and second body segment, and where the second body segment is in a predetermined pose, aligned in an arbitrary direction relative to Earth;
simultaneous with measuring the primary IMU sensor's first orientation, measuring an auxiliary IMU sensor orientation; and,
calculating an auxiliary alignment orientation relationship between the auxiliary IMU sensor orientation and the second body segment orientation.

9. The method of claim 1 wherein measuring the primary IMU sensor orientation includes:
aligning an EROMD with the first body segment; and,
simultaneous with measuring the primary IMU sensor orientation, measuring an EROMD orientation with the first body segment in an arbitrary pose, in an arbitrary direction relative to Earth.

10. The method of claim 1 wherein measuring the primary IMU sensor orientation includes estimating the alignment orientation relationship between the primary IMU sensor orientation and the first body segment orientation;
wherein calculating the alignment orientation relationship between the primary IMU sensor orientation and the first body segment orientation includes:
using a body segment musculoskeletal model describing potential movement relationships between adjacent body segments to find deterministic, axial rotation, and radial rotation limits associated with the first body segment, where deterministic limits describe the likely accuracy of the estimated alignment orientation relationship;
in response to comparing the deterministic, axial rotation, and radial rotation limits with subsequent calculated movement relationships between the first body segment and an adjacent body segment, updating the estimated alignment orientation relationship.

11. A method for determining separate constituent axial and radial rotations of a connected joint, the method comprising:
providing a joint connecting a distal body segment to a proximal body segment;
monitoring a composite joint rotation;
applying a musculoskeletal model of the joint to the monitored joint rotation, where the model permits only decompositions with physiologically possible constituent rotations; and,
calculating axial and radial rotations of the distal body segment relative to the proximal body segment.

12. A system for determining the orientation of a body segment using an inertia measurement unit (IMU) sensor capable of measuring its orientation relative to Earth, the system comprising:
a primary IMU sensor mounted on a first body segment and having an output to supply signals associated with an unknown first alignment orientation relationship between the primary IMU sensor and the first body segment;
a processor;
a non-transitory memory; and,
an alignment application embedded in the non-transitory memory including a sequence of processor executable instructions for accepting the primary IMU sensor signals, measuring a primary IMU sensor orientation, and calculating an alignment orientation relationship between the primary IMU sensor orientation and a first body segment orientation.

13. The system of claim 12 wherein the alignment application measures a primary IMU sensor initial orientation and a subsequent orientation, and determines a subsequent orientation of the first body segment in response to the primary IMU sensor initial and subsequent orientations, and the calculation of the alignment orientation relationship between the primary IMU sensor initial orientation and the first body segment orientation.

14. The system of claim 13 further comprising:
a body segment musculoskeletal model, stored in the non-transitory memory, describing potential movement relationships between adjacent body segments;
wherein the alignment application determines the subsequent orientation of first body segment using the musculoskeletal model to find deterministic, axial rotation, and radial rotation limits associated with the first body segment, where deterministic limits describe the likely accuracy of a first alignment orientation relationship estimate; and,
wherein the alignment application has an interface for alerting a user when the estimated relationship between adjacent body segments exceeds the limits of the musculoskeletal model.

15. The system of claim 12 wherein the alignment application measures the primary IMU sensor orientation with the first body segment in a predetermined pose, aligned in a predetermined direction relative to Earth.

16. The system of claim 12 further comprising:
an Earth relative orientation measurement device (EROMD) having an output to supply signals associated with its current orientation relative to Earth, aligned with a predetermined second body segment in a predetermined pose, in an arbitrary direction relative to Earth; and,
wherein the alignment application, simultaneous with measuring the primary IMU sensor orientation, measures the EROMD orientation.

17. The system of claim 12 further comprising:
an auxiliary IMU sensor having an output to supply signals associated with being mounted on a second body segment, where an alignment orientation relationship between the auxiliary IMU sensor and second body segment is known;
wherein the alignment application has an interface to accept a measurement of the first body segment orientation with respect to a second body segment found using a goniometer; and,
wherein the alignment application measures the primary IMU sensor orientation by simultaneously measuring the primary IMU sensor orientation and the auxiliary IMU sensor orientation.

18. The system of claim 12 wherein the alignment application measures a primary IMU sensor first orientation with the first body segment in a predetermined pose, aligned in an arbitrary direction relative to Earth, and measures a primary IMU sensor second orientation with the first body segment moving in a predetermined manner.

19. The system of claim 18 further comprising:
an auxiliary IMU sensor having an output to supply signals associated with being mounted on a second body segment with an unknown second alignment orientation relationship between the auxiliary IMU sensor and second body segment, and where the second body segment is in a predetermined pose, aligned in an arbitrary direction relative to Earth; and,
wherein the alignment application simultaneous with measuring the primary IMU sensor's first orientation, measures the auxiliary IMU sensor orientation, and calculates an auxiliary alignment orientation relationship between the auxiliary IMU sensor orientation and the second body segment orientation.

20. The system of claim 12 further comprising:
an EROMD having an output to supply signals associated with its current orientation relative to Earth, aligned with the first body segment in an arbitrary pose, in an arbitrary direction relative to Earth; and,
wherein the alignment application simultaneously measures the primary IMU sensor orientation and the EROMD orientation.

21. The system of claim 12 further comprising:
a body segment musculoskeletal model, stored in the non-transitory memory, describing potential movement relationships between adjacent body segments;
wherein the alignment application estimates the alignment orientation relationship between the primary IMU sensor orientation and the first body segment orientation; and,
wherein the alignment application calculates the alignment orientation relationship between the primary IMU sensor orientation and the first body segment orientation by using the body segment musculoskeletal model to find deterministic, axial rotation, and radial rotation limits associated with the first body segment, where deterministic limits describe the likely accuracy of the estimated alignment orientation relationship, compares the deterministic, axial rotation, and radial rotation limits with subsequent calculated movement relationships between the first body segment and an adjacent body segment, and updates the estimated alignment orientation relationship.

22. The system of claim 12 further comprising:
a body segment musculoskeletal model, stored in the non-transitory memory, describing physiologically possible constituent rotations for a first joint connecting two adjoining body segments; and,
wherein the alignment application determines separate constituent axial and radial rotations for the first joint by applying the musculoskeletal model.
